SEQUENCE LISTING (146 sequences)

SEQ ID NO: 1 - DNA, 60 bp, Artificial Sequence
  Synthetic construct
aaataattttgtttaactttaagaaggagatataccatgaaagttaaagatctgcgtaaa

SEQ ID NO: 2 - DNA, 70 bp, Artificial Sequence
  Synthetic construct
cctttcgggctttgttagcagccggatcctcgattagtgatgatgatgatgatggtatagttcatccatg

SEQ ID NO: 3 - DNA, 60 bp, Artificial Sequence
  Synthetic construct
aaataattttgtttaactttaagaaggagatataccatgcgtaaaggagaagaacttttc

SEQ ID NO: 4 - DNA, 70 bp, Artificial Sequence
  Synthetic construct
tcctttcgggctttgttagcagccggatcctcgattagtgatgatgatgatgatgtttgtatagttcatc

SEQ ID NO: 5 - DNA, 60 bp, Artificial Sequence
  Synthetic construct
aaataattttgtttaactttaagaaggagatataccatggttagtaaaggagaagaaaac

SEQ ID NO: 6 - DNA, 70 bp, Artificial Sequence
  Synthetic construct
cctttcgggctttgttagcagccggatcctcgattagtgatgatgatgatgatgtgcggtaccagaacct

SEQ ID NO: 7 - DNA, 50 bp, Artificial Sequence
  Synthetic construct
ctttaagaaggagatataccatgcaatggttagtaaaggagaagaaaaca

SEQ ID NO: 8 - DNA, 60 bp, Artificial Sequence
  Synthetic construct
aaataattttgtttaactttaagaaggagatataccatggtgagcaagggcgaggaggat

SEQ ID NO: 9 - DNA, 70 bp, Artificial Sequence
  Synthetic construct
cctttcgggctttgttagcagccggatcctcgattagtgatgatgatgatgatgcttgtacagctcgtcc

SEQ ID NO: 10 - DNA, 59 bp, Artificial Sequence
  Synthetic construct
cggataacaattcccctctagactacaacttcttctcaatcagaaggagtaagtcaatg

SEQ ID NO: 11 - DNA, 60 bp, Artificial Sequence
  Synthetic construct
caccataaccgaaagtagtgacaagtgttggccatggaacaggtagttttccagtagtgc

SEQ ID NO: 12 - DNA, 60 bp, Artificial Sequence
  Synthetic construct
gtatatctccttcttaaagttaaacaaaattatttctagaggggaattgttatccgctca

SEQ ID NO: 13 - DNA, 60 bp, Artificial Sequence
  Synthetic construct
gtttaactttaagaaggagatataccatgcatcatcatcaccatcatttaattaaagcag

SEQ ID NO: 14 - DNA, 60 bp, Artificial Sequence
  Synthetic construct
gctttgttagcagccggatcctcgagcatatggccgctgctttaattaaatgatggtgat

SEQ ID NO: 15 - DNA, 60 bp, Artificial Sequence
  Synthetic construct
tgcaggagtcgcataagggagagcgtcgacccctggatgctgtaggcataggcttggtta

SEQ ID NO: 16 - DNA, 60 bp, Artificial Sequence
  Synthetic construct
tccatgccaacccgttccatgtgctcgccggatcccgcgaaattaatacgactcactata

SEQ ID NO: 17 - DNA, 60 bp, Artificial Sequence
  Synthetic construct
gtttgcgggcagcaaaacccgtaccctaggccctggatgctgtaggcataggcttggtta

SEQ ID NO: 18 - DNA, 60 bp, Artificial Sequence
  Synthetic construct
atcacgaggccctttcgtcttcacctcgatgatcccgcgaaattaatacgactcactata

SEQ ID NO: 19 - DNA, 60 bp, Artificial Sequence
  Synthetic construct
tataccatgcatcatcatcaccatcatttaagcaagagcaccgctgagatccgtcaggcg

SEQ ID NO: 20 - DNA, 53 bp, Artificial Sequence
  Synthetic construct
agcatatggccgctgctttaatattgcaatttcgcgctgacccagcctttcac

SEQ ID NO: 21 - DNA, 60 bp, Artificial Sequence
  Synthetic construct
tataccatgcatcatcatcaccatcatttaaatattcaggctcttctctcagaaaaagtc

SEQ ID NO: 22 - DNA, 53 bp, Artificial Sequence
  Synthetic construct
agcatatggccgctgctttaatacatacgctctacagtctcaatacccagcgt

SEQ ID NO: 23 - DNA, 60 bp, Artificial Sequence
  Synthetic construct
tataccatgcatcatcatcaccatcatttaagcgttgtgcctgtagccgacgtactccag

SEQ ID NO: 24 - DNA, 50 bp, Artificial Sequence
  Synthetic construct
tggtgttggagttttcagcacggaaagtcgggccgaaggtataaattttg

SEQ ID NO: 25 - DNA, 50 bp, Artificial Sequence
  Synthetic construct
tttccgtgctgaaaactccaacaccagccgtcacctggcggaattctgga

SEQ ID NO: 26 - DNA, 53 bp, Artificial Sequence
  Synthetic construct
agcatatggccgctgctttaatagaagctggcgttacgcggagtacgtgggaa

SEQ ID NO: 27 - DNA, 60 bp, Artificial Sequence
  Synthetic construct
ttaactttaagaaggagatataccatgcatcgtacagaatattgtggacagctccgtttg

SEQ ID NO: 28 - DNA, 60 bp, Artificial Sequence
  Synthetic construct
accggcgacgaaattaatgatggtgatgatgatggttattctcagccttcttcacaacct

SEQ ID NO: 29 - DNA, 60 bp, Artificial Sequence
  Synthetic construct
ttaactttaagaaggagatataccatgcatctaaaaatcttcaatactctgacacgccaa

SEQ ID NO: 30 - DNA, 60 bp, Artificial Sequence
  Synthetic construct
accggcgacgaaattaatgatggtgatgatgatgcttacgacgccaggtggtcccttgcg

SEQ ID NO: 31 - DNA, 60 bp, Artificial Sequence
  Synthetic construct
ttaactttaagaaggagatataccatgcattcagaatcactacgtattatttttgcgggt

SEQ ID NO: 32 - DNA, 60 bp, Artificial Sequence
  Synthetic construct
accggcgacgaaattaatgatggtgatgatgatggaccagacggttgcccggaacaaacc

SEQ ID NO: 33 - DNA, 60 bp, Artificial Sequence
  Synthetic construct
ttaactttaagaaggagatataccatgcatattagcgatatcagaaaagatgctgaagta

SEQ ID NO: 34 - DNA, 60 bp, Artificial Sequence
  Synthetic construct
accggcgacgaaattaatgatggtgatgatgatggaactgcatcagttctgcttctttgt

SEQ ID NO: 35 - DNA, 60 bp, Artificial Sequence
  Synthetic construct
ttaactttaagaaggagatataccatgcatgctcgtacaacacccatcgcacgctaccgt

SEQ ID NO: 36 - DNA, 60 bp, Artificial Sequence
  Synthetic construct
accggcgacgaaattaatgatggtgatgatgatgtttaccacgggcttcaattacggcct

SEQ ID NO: 37 - DNA, 60 bp, Artificial Sequence
  Synthetic construct
tataccatgcatcatcatcaccatcatttaagtgaggcagaagcccgcccgactaacttt

SEQ ID NO: 38 - DNA, 53 bp, Artificial Sequence
  Synthetic construct
agcatatggccgctgctttaatactcgcctactttcgcccaggtatcacgcag

SEQ ID NO: 39 - DNA, 60 bp, Artificial Sequence
  Synthetic construct
ttaactttaagaaggagatataccatgcataaaatcaaaactcgcttcgcgccaagccca

SEQ ID NO: 40 - DNA, 60 bp, Artificial Sequence
  Synthetic construct
accggcgacgaaattaatgatggtgatgatgatgctgctgattttcgcgttcagcaataa

SEQ ID NO: 41 - DNA, 60 bp, Artificial Sequence
  Synthetic construct
ttaactttaagaaggagatataccatgcatcaaaagtttgataccaggaccttccagggc

SEQ ID NO: 42 - DNA, 60 bp, Artificial Sequence
  Synthetic construct
accggcgacgaaattaatgatggtgatgatgatgcttatctttgttgcacatcgggaagc

SEQ ID NO: 43 - DNA, 60 bp, Artificial Sequence
  Synthetic construct
ttaactttaagaaggagatataccatgcattctgagaaaacttttctggtggaaatcggc

SEQ ID NO: 44 - DNA, 60 bp, Artificial Sequence
  Synthetic construct
accggcgacgaaattaatgatggtgatgatgatgttgcaacagcgaaatatccgcaacgc

SEQ ID NO: 45 - DNA, 60 bp, Artificial Sequence
  Synthetic construct
ttaactttaagaaggagatataccatgcatgcaaaaaacattcaagccattcgcggcatg

SEQ ID NO: 46 - DNA, 60 bp, Artificial Sequence
  Synthetic construct
accggcgacgaaattaatgatggtgatgatgatgacccagtaacgtgcgcaaatgcgcgg

SEQ ID NO: 47 - DNA, 60 bp, Artificial Sequence
  Synthetic construct
tataccatgcatcatcatcaccatcatttaagtgactataaatcaaccctgaatttgccg

SEQ ID NO: 48 - DNA, 53 bp, Artificial Sequence
  Synthetic construct
agcatatggccgctgctttaataggcaaacttacgtttttcaccgtcaccggc

SEQ ID NO: 49 - DNA, 60 bp, Artificial Sequence
  Synthetic construct
tataccatgcatcatcatcaccatcatttagccaaagaagacaatattgaaatgcaaggt

SEQ ID NO: 50 - DNA, 53 bp, Artificial Sequence
  Synthetic construct
agcatatggccgctgctttaatagcgactacggaagacaatgcggcctttgct

SEQ ID NO: 51 - DNA, 60 bp, Artificial Sequence
  Synthetic construct
tataccatgcatcatcatcaccatcatttaacagatgtaacgattaaaacgctggccgca

SEQ ID NO: 52 - DNA, 53 bp, Artificial Sequence
  Synthetic construct
agcatatggccgctgctttaataagcaatggtacgttggatctcgatgatttc

SEQ ID NO: 53 - DNA, 60 bp, Artificial Sequence
  Synthetic construct
tataccatgcatcatcatcaccatcatttaaaaggcggaaaacgagttcaaacggcgcgc

SEQ ID NO: 54 - DNA, 53 bp, Artificial Sequence
  Synthetic construct
agcatatggccgctgctttaatactgtttcttcttaggagcgagcaccatgat

SEQ ID NO: 55 - DNA, 60 bp, Artificial Sequence
  Synthetic construct
ttaactttaagaaggagatataccatgcataagaatatacgtaacttttcgatcatagct

SEQ ID NO: 56 - DNA, 60 bp, Artificial Sequence
  Synthetic construct
accggcgacgaaattaatgatggtgatgatgatgtttgttgtctttgccgacgtgcagaa

SEQ ID NO: 57 - DNA, 60 bp, Artificial Sequence
  Synthetic construct
ttaactttaagaaggagatataccatgcatcaagagcaataccgcccggaagagatagaa

SEQ ID NO: 58 - DNA, 60 bp, Artificial Sequence
  Synthetic construct
accggcgacgaaattaatgatggtgatgatgatggccaacgaccagattgaggagtttac

SEQ ID NO: 59 - DNA, 60 bp, Artificial Sequence
  Synthetic construct
ttaactttaagaaggagatataccatgcattctgaacaacacgcacagggcgctgacgcg

SEQ ID NO: 60 - DNA, 60 bp, Artificial Sequence
  Synthetic construct
accggcgacgaaattaatgatggtgatgatgatgttttaccggacgcatcgccgggaaca

SEQ ID NO: 61 - DNA, 60 bp, Artificial Sequence
  Synthetic construct
ttaactttaagaaggagatataccatgcatactcaagtcgcgaagaaaattctggtgacg

SEQ ID NO: 62 - DNA, 60 bp, Artificial Sequence
  Synthetic construct
accggcgacgaaattaatgatggtgatgatgatgtttcacctgatgacccggtttagcac

SEQ ID NO: 63 - DNA, 60 bp, Artificial Sequence
  Synthetic construct
tataccatgcatcatcatcaccatcatttatcacatctcgcagaactggttgccagtgcg

SEQ ID NO: 64 - DNA, 53 bp, Artificial Sequence
  Synthetic construct
agcatatggccgctgctttaatatttaaactgtttgaggaaacgcagatcgtt

SEQ ID NO: 65 - DNA, 60 bp, Artificial Sequence
  Synthetic construct
ttaactttaagaaggagatataccatgcataaattcagtgaactgtggttacgcgaatgg

SEQ ID NO: 66 - DNA, 60 bp, Artificial Sequence
  Synthetic construct
accggcgacgaaattaatgatggtgatgatgatgatccctcaatgatgcctggaatcgct

SEQ ID NO: 67 - DNA, 60 bp, Artificial Sequence
  Synthetic construct
tataccatgcatcatcatcaccatcatttaaagccttctatcgttgccaaactggaagcc

SEQ ID NO: 68 - DNA, 53 bp, Artificial Sequence
  Synthetic construct
agcatatggccgctgctttaatattcctgctcggacaacgccgccagttggtc

SEQ ID NO: 69 - DNA, 60 bp, Artificial Sequence
  Synthetic construct
tataccatgcatcatcatcaccatcatttatttgaaattaatccggtaaataatcgcatt

SEQ ID NO: 70 - DNA, 53 bp, Artificial Sequence
  Synthetic construct
agcatatggccgctgctttaatataaccctgctttcaaacttgcttcgataaa

SEQ ID NO: 71 - DNA, 60 bp, Artificial Sequence
  Synthetic construct
tataccatgcatcatcatcaccatcatttaacgttgtctccttatttgcaagaggtggcg

SEQ ID NO: 72 - DNA, 53 bp, Artificial Sequence
  Synthetic construct
agcatatggccgctgctttaataatgctcgcgggtctggtggaactgaacgtc

SEQ ID NO: 73 - DNA, 60 bp, Artificial Sequence
  Synthetic construct
ttaactttaagaaggagatataccatgcatcgtactagccaatacctgctctccactctc

SEQ ID NO: 74 - DNA, 60 bp, Artificial Sequence
  Synthetic construct
accggcgacgaaattaatgatggtgatgatgatggcctttaatctgtttcaccagatatt

SEQ ID NO: 75 - DNA, 60 bp, Artificial Sequence
  Synthetic construct
ttaactttaagaaggagatataccatgcatctcgatcccaatctgctgcgtaatgagcca

SEQ ID NO: 76 - DNA, 60 bp, Artificial Sequence
  Synthetic construct
accggcgacgaaattaatgatggtgatgatgatggccaatatattccagtccgttcatat

SEQ ID NO: 77 - DNA, 60 bp, Artificial Sequence
  Synthetic construct
tataccatgcatcatcatcaccatcatttacctgttataactcttcctgatggcagccaa

SEQ ID NO: 78 - DNA, 53 bp, Artificial Sequence
  Synthetic construct
agcatatggccgctgctttaatattcctccaattgtttaagactgcggctgcg

SEQ ID NO: 79 - DNA, 60 bp, Artificial Sequence
  Synthetic construct
ttaactttaagaaggagatataccatgcatactaagcccatcgtttttagtggcgcacag

SEQ ID NO: 80 - DNA, 60 bp, Artificial Sequence
  Synthetic construct
accggcgacgaaattaatgatggtgatgatgatgcggcttcgccacaaaaccaatcgctt

SEQ ID NO: 81 - DNA, 60 bp, Artificial Sequence
  Synthetic construct
ttaactttaagaaggagatataccatgcatgctgaaattaccgcatccctggtaaaagag

SEQ ID NO: 82 - DNA, 60 bp, Artificial Sequence
  Synthetic construct
accggcgacgaaattaatgatggtgatgatgatgagactgcttggacatcgcagcaactt

SEQ ID NO: 83 - DNA, 60 bp, Artificial Sequence
  Synthetic construct
ttaactttaagaaggagatataccatgcattctaaagaaaaatttgaacgtacaaaaccg

SEQ ID NO: 84 - DNA, 60 bp, Artificial Sequence
  Synthetic construct
accggcgacgaaattaatgatggtgatgatgatggcccagaactttagcaacaacgcccg

SEQ ID NO: 85 - DNA, 60 bp, Artificial Sequence
  Synthetic construct
ttaactttaagaaggagatataccatgcatgcaagcagtaacttgattaaacaattgcaa

SEQ ID NO: 86 - DNA, 60 bp, Artificial Sequence
  Synthetic construct
accggcgacgaaattaatgatggtgatgatgatgtttccagcaaatcagacagtaattct

SEQ ID NO: 87 - DNA, 60 bp, Artificial Sequence
  Synthetic construct
ttaactttaagaaggagatataccatgcatgaaaagacatataacccacaagatatcgaa

SEQ ID NO: 88 - DNA, 60 bp, Artificial Sequence
  Synthetic construct
accggcgacgaaattaatgatggtgatgatgatgcagcgcggcgataacagcctgctgtt

SEQ ID NO: 89 - DNA, 46 bp, Artificial Sequence
  Synthetic construct
gatcctcgagcatatggccgctgctttaatgatggtgatgatgatg

SEQ ID NO: 90 - DNA, 60 bp, Artificial Sequence
  Synthetic construct
attgtgagcggataacaattcccctctagacccaggtctatacgtagtaaggaggtaagg

SEQ ID NO: 91 - DNA, 60 bp, Artificial Sequence
  Synthetic construct
cgggttgctcggcagctgaatttccaccagttcgcccaccgccacggtcagggtcgcgcc

SEQ ID NO: 92 - DNA, 60 bp, Artificial Sequence
  Synthetic construct
ctggtggaaattcagctgccgagcaacccgaccaccggctttgcgtggtattttgaaggc

SEQ ID NO: 93 - DNA, 60 bp, Artificial Sequence
  Synthetic construct
ctggtggaaattcagctgccgagcaacccgggcaccggctttgcgtggtattttgaaggc

SEQ ID NO: 94 - DNA, 60 bp, Artificial Sequence
  Synthetic construct
tttgctatccggcggaaaatatttgttttccacggtaaacatgctttcgttcgggctttc

SEQ ID NO: 95 - DNA, 60 bp, Artificial Sequence
  Synthetic construct
caaatattttccgccggatagcaaavkgbkkggckbsggcggcaccgaacattttcatgt

SEQ ID NO: 96 - DNA, 45 bp, Artificial Sequence
  Synthetic construct
catataggtcaggttcaccgcatgggtgcccgccgctttcacggt

SEQ ID NO: 97 - DNA, 60 bp, Artificial Sequence
  Synthetic construct
gggcacccatgcggtgaacctgacctatatgcrrshgtkkaccggcccgagccatvmtag

SEQ ID NO: 98 - DNA, 60 bp, Artificial Sequence
  Synthetic construct
aacgactacaaagacgatgacgacaagtaaaagcttctttcagcaaaaaaccccgcgaga

SEQ ID NO: 99 - protein, 13 aa, Trypanosoma cruzi
QLPSNPTTGFAWY

SEQ ID NO: 100 - protein, 13 aa, Trypanosoma cruzi
TLTVAVGELVEIQ

SEQ ID NO: 101 - protein, 13 aa, Unknown
  Leishmania major strain Friedlin
HLEGNPTTGYTWT

SEQ ID NO: 102 - protein, 13 aa, Trypanosoma brucei
ELESNPTTGYTWL

SEQ ID NO: 103 - protein, 13 aa, Trypanosoma brucei
ELKSNPTTGYTWL

SEQ ID NO: 104 - protein, 13 aa, Leishmania infantum
HLKGNPTTGYMWT

SEQ ID NO: 105 - protein, 13 aa, Leishmania panamensis
VLNGNPTTGYTWT

SEQ ID NO: 106 - protein, 13 aa, Leishmania braziliensis
LLNGNPTTGYTWT

SEQ ID NO: 107 - protein, 13 aa, Entamoeba nuttalli
ILPGNPTTGYSWV

SEQ ID NO: 108 - protein, 13 aa, Entamoeba histolytica
ILHGNPTTGYSWV

SEQ ID NO: 109 - protein, 13 aa, Leishmania mexicana
HLKGNPTTGYMWT

SEQ ID NO: 110 - protein, 13 aa, Pseudomonas aeruginosa
VLTLPSNPTTGFR

SEQ ID NO: 111 - protein, 13 aa, Pseudomonas mendocina
SLPSNPTTGFRWE

SEQ ID NO: 112 - protein, 13 aa, Bacillus cereus
TVTMSIGDIFSIQ

SEQ ID NO: 113 - protein, 13 aa, Plasmodium berghei
DLEGNAGTGYLWV

SEQ ID NO: 114 - protein, 8 aa, Trypanosoma cruzi
DSKLLGAG

SEQ ID NO: 115 - protein, 8 aa, Trypanosoma cruzi
FPPDSKLL

SEQ ID NO: 116 - protein, 8 aa, Unknown
  Leishmania major strain Friedlin
SGSMVGAG

SEQ ID NO: 117 - protein, 8 aa, Trypanosoma brucei
RRLLVGAG

SEQ ID NO: 118 - protein, 8 aa, Trypanosoma brucei
RRLLVGAG

SEQ ID NO: 119 - protein, 8 aa, Leishmania infantum
SGPMVGVG

SEQ ID NO: 120 - protein, 8 aa, Leishmania panamensis
PSGMVGVG

SEQ ID NO: 121 - protein, 8 aa, Leishmania braziliensis
PSGMVGVG

SEQ ID NO: 122 - protein, 8 aa, Entamoeba nuttalli
APGICGCG

SEQ ID NO: 123 - protein, 8 aa, Entamoeba histolytica
APGICGCG

SEQ ID NO: 124 - protein, 8 aa, Leishmania mexicana
STPMVGVG

SEQ ID NO: 125 - protein, 8 aa, Pseudomonas aeruginosa
DSGLVGSG

SEQ ID NO: 126 - protein, 8 aa, Pseudomonas mendocina
DAGLVGAG

SEQ ID NO: 127 - protein, 8 aa, Bacillus cereus
SNNGIGSS

SEQ ID NO: 128 - protein, 10 aa, Plasmodium berghei
KPQMQLLGGP

SEQ ID NO: 129 - protein, 14 aa, Trypanosoma cruzi
TYMRPWTGPSHDSE

SEQ ID NO: 130 - protein, 14 aa, Trypanosoma cruzi
TYMRPWAGPSHDSE

SEQ ID NO: 131 - protein, 14 aa, Unknown
  Leishmania major strain Friedlin
VYARPFEGPKPDNE

SEQ ID NO: 132 - protein, 14 aa, Trypanosoma brucei
AYGRPWVGFNAAAK

SEQ ID NO: 133 - protein, 14 aa, Trypanosoma brucei
AYGRPWVGFNAAAK

SEQ ID NO: 134 - protein, 14 aa, Leishmania infantum
VYMRPFEGLKPENE

SEQ ID NO: 135 - protein, 14 aa, Leishmania panamensis
LYIRPFEGLKLDSQ

SEQ ID NO: 136 - protein, 14 aa, Leishmania braziliensis
VYIRLFEGIKLDSQ

SEQ ID NO: 137 - protein, 13 aa, Entamoeba nuttalli
VYRRPWAPNANDR

SEQ ID NO: 138 - protein, 13 aa, Entamoeba histolytica
VYRRPWAPNANDR

SEQ ID NO: 139 - protein, 14 aa, Leishmania mexicana
VYTRPFEGIKPENE

SEQ ID NO: 140 - protein, 14 aa, Pseudomonas aeruginosa
VYRRPWEKDAEPAE

SEQ ID NO: 141 - protein, 14 aa, Pseudomonas mendocina
TYQQPWEPNVPPAQ

SEQ ID NO: 142 - protein, 14 aa, Bacillus cereus
KNWQEWSGEASIVD

SEQ ID NO: 143 - protein, 13 aa, Plasmodium berghei
SYYRPFSPTSGAN

SEQ ID NO: 144 - protein, 13 aa, Unknown
  Consensus sequence
  MISC_FEATURE (7)..(7): Xaa = Thr or Gly
QLPSNPXTGFAWY

SEQ ID NO: 145 - protein, 8 aa, Unknown
  Consensus sequence
  MISC_FEATURE (4)..(4): Xaa = Gly, Leu, Met, Arg, or Val
  MISC_FEATURE (5)..(5): Xaa = Cys, Phe, Gly, Leu, Arg, Val, or Trp
  MISC_FEATURE (7)..(7): Xaa = Ala, Cys, Phe, Gly, Leu, Ser, Val, or Trp
DSKXXGXG

SEQ ID NO: 146 - protein, 13 aa, Unknown
  Consensus sequence
  MISC_FEATURE (4)..(4): Xaa = Gln or Arg
  MISC_FEATURE (5)..(5): Xaa = Ala, Glu, Leu, Pro, Gln, or Val
  MISC_FEATURE (6)..(6): Xaa = Cys, Phe, Leu, or Trp
  MISC_FEATURE (12)..(12): Xaa = Ala, Asp, His, Asn, Pro, or Thr
TYMXXXTGPSHXS

(19) United States
(12) Patent Application Publication
(10) Pub. No.: US 2019/0376069 A1
(43) Pub. Date: Dec. 12, 2019

(54) USE OF MICROBIAL CONSORTIA IN THE PRODUCTION OF MULTI-PROTEIN COMPLEXES

(71) Applicant: The Regents of the University of California, Oakland, CA (US)

(72) Inventors: Fernando VILLARREAL, Davis, CA (US); Cheemeng TAN, Davis, CA (US)

(21) Appl. No.: 16/483,979

(22) PCT Filed: Feb. 6, 2018
(86) PCT No.: PCT/US2018/017102

§ 371 Date: Aug. 6, 2019

Related U.S. Application Data
(60) Provisional application No. 62/455,941, filed on Feb. 7, 2017.

(51) Int. Cl. C12N 15/70; C12P 21/00; C12N 1/20; C12N 15/62; C07K 14/47

(52) U.S. Cl. CPC C12N 15/70; C12P 21/00; C12N 1/20; C07K 2319/21; C07K 14/4702; C07K 2319/00; C12N 15/62

(57) ABSTRACT

The present invention provides microbial cultures (referred to here as microbial consortia) comprising a plurality of microbial strains each strain comprising a different recombinant plasmid including a gene encoding a different protein involved in translation of mRNA. The protein expression level of each protein is controlled to a pre-defined level, such that the proteins are capable of forming a multi-protein complex which translates an mRNA molecule into a polypeptide in a reaction mixture.

### CROSS-REFERENCE TO RELATED PATENT APPLICATIONS

The present patent application claims benefit of priority to U.S. Provisional Patent Application No. 62/455,941, filed Feb. 7, 2017, of which are incorporated by reference for all purposes.

### STATEMENT AS TO RIGHTS TO INVENTIONS MADE UNDER FEDERALLY SPONSORED RESEARCH AND DEVELOPMENT

### Field of the Invention

This invention relates to microbial consortia and their use in production of multi-protein complexes.

### Background of the Invention

Protein purification is conducted routinely in areas encompassing biochemical characterization of cellular pathways (Goering et al., 2016; Lu et al., 2015; Shimizu and Ueda, 2010) to in vitro, cell-free assays (Caschera and Noireaux, 2016; Niederholtmeyer et al., 2015; Pardee et al., 2014; Takahashi et al., 2015; Tsuji et al., 2016). While the classical approach works well for the synthesis of one protein species, the preparation of multi-protein complexes, especially in the case of metabolic pathways (Lopez-Gallego and Schmidt-Dannert, 2010) and mRNA translation machinery (TraM) (Shimizu and Ueda, 2010), remains difficult due to the large number of protein species and stringent requirement of protein ratios (Li et al., 2014; Matsubayashi and Ueda, 2014). TraM consists of 34 proteins, including 11 IET genes (3 Initiation factors, 4 Elongation factors, 3 Termination/Release factors and the Ribosome Recycling Factor), and 23 AAT (tRNA-Amino acyl-transferases) (Shimizu and Ueda, 2010). Pure TraM proteins are traditionally prepared by purifying each protein individually or few proteins at a time, and then mixing them to assemble the functional TraM (Shimizu and Ueda, 2010; Wang et al., 2012).

There is a need in the art for new methods of providing the proteins required for in vitro translation. The present invention addresses these and other needs.

### BRIEF SUMMARY OF THE INVENTION

The present invention provides a microbial culture (referred to here as a microbial consortium) comprising a plurality of microbial strains, each strain comprising a different recombinant plasmid including a gene encoding a different protein involved in translation of mRNA, wherein the protein expression level of each protein is controlled to a pre-defined level, such that the proteins are capable of forming a multi-protein complex which translates an mRNA molecule into a polypeptide in a reaction mixture.

The amount of each protein can be determined by: (a) the density of the microbial strain in the culture, (b) the copy number of the plasmid comprising the gene encoding the protein, (c) the sequence of the ribosomal binding site in the gene encoding the protein; or (d) a combination of (a), (b) and (c). Each protein in the multi-protein complex may include a tag to facilitate isolation of the protein (e.g., poly His tag).

In a typical embodiment, each gene has the same promoter (e.g., a PT7/lacO hybrid promoter) and the microbial culture comprises E. coli. Each microbial strain may comprise a single plasmid including a gene encoding a protein involved in translation of mRNA. Alternatively, at least one strain comprises more than one plasmid including a gene encoding a protein involved in translation of mRNA.

The proteins in the multi-protein complex may comprise initiation factors, elongation factors, termination/release factors, a ribosome recycling factor and tRNA-Amino acyl-transferases. In some embodiments, the initiation factors are translational initiation factor 1, translational initiation factor 2, and translational initiation factor 3; the elongation factors are translational elongation factor G, translational elongation factor Tu, translational elongation factor Ts, and translational elongation factor 4; the termination/release factors are translational release factor 1, translational release factor 2, and translational release factor 3; and the tRNA-Amino acyl-transferases are Val-tRNA synthetase, Met-tRNA synthetase, Ile-tRNA synthetase, Thr-tRNA synthetase, Lys-tRNA synthetase, Glu-tRNA synthetase, Ala-tRNA synthetase, Asp-tRNA synthetase, Asn-tRNA synthetase, Leu-tRNA synthetase, Arg-tRNA synthetase, Cys-tRNA synthetase, Trp-tRNA synthetase, Phe-tRNA synthetase B, Pro-tRNA synthetase, Ser-tRNA synthetase, Phe-tRNA synthetase A, Gln-tRNA synthetase, Tyr-tRNA synthetase, Met-tRNA formyltransferase, Gly-tRNA synthetase B, His-tRNA synthetase, and Gly-tRNA synthetase A.

The invention also provides methods of making a multi-protein complex as described above. The methods comprise (a) providing a microbial culture comprising a plurality of microbial strains, each strain comprising a different recombinant plasmid including a gene encoding a different protein involved in translation of mRNA, wherein the protein expression level of each protein is controlled to a pre-defined level, such that the proteins are capable of forming a multi-protein complex; and (b) simultaneously isolating the proteins from the microbial culture, thereby forming the multi-protein complex.

The invention further provides methods of translating an mRNA molecule into a polypeptide. The methods comprise: (a) providing a microbial culture comprising a plurality of microbial strains, each strain comprising a different recombinant plasmid comprising a gene encoding a different protein involved in translation of mRNA, wherein the protein expression level of each protein is controlled to a pre-defined level, such that the proteins are capable of forming a multi-protein complex which translates an mRNA molecule into a polypeptide in a reaction mixture; (b) simultaneously isolating the proteins from the microbial culture, thereby forming the multi-protein complex; (c) forming a reaction mixture comprising the multi-protein complex, amino acids, ribosomes, and the mRNA molecule or a DNA molecule encoding the mRNA; (d) incubating the reaction mixture under conditions suitable for translation of the mRNA molecule into a polypeptide; and (e) isolating the polypeptide.

### Definitions

“Operably linked” indicates that two or more DNA segments are joined together such that they function in concert for their intended purposes. For example, coding sequences are operably linked to promoter in the correct reading frame such that transcription initiates in the promoter and proceeds through the coding segment(s) to the terminator.

A “polynucleotide” is a single- or double-stranded polymer of deoxyribonucleotide or ribonucleotide bases typically read from the 5′ to the 3′ end. Polynucleotides include RNA and DNA, and may be isolated from natural sources, synthesized in vitro, or prepared from a combination of natural and synthetic molecules. When the term is applied to double-stranded molecules it is used to denote overall length and will be understood to be equivalent to the term “base pairs”.

A “polypeptide” or “protein” is a polymer of amino acid residues joined by peptide bonds, whether produced naturally or synthetically. Polypeptides of less than about 75 amino acid residues are also referred to here as peptides or oligopeptides.

The term “promoter” is used herein for its art-recognized meaning to denote a portion of a gene containing DNA sequences that provide for the binding of RNA polymerase and initiation of transcription of an operably linked coding sequence. Promoter sequences are typically found in the 5′ non-coding regions of genes.

The terms “identical” or percent “identity,” in the context of two or more nucleic acids or polypeptide sequences, (e.g., two proteins of the invention and polynucleotides that encode them) refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides of the invention, refers to two or more sequences or subsequences that have at least 60%, 65%, 70%, 75%, 80%, or 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides of the invention are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

### BRIEF DESCRIPTION OF THE DRAWINGS

FIG. 1. Basic mechanisms that control protein co-expression and co-purification from a single bacterial consortium. (A) Four strains expressing 6x-His tagged CFP, GFP, mOrange, and mCherry are used to investigate protein co-expression levels in the consortia and co-purification using one-shot strategy. A mathematical model is also used to predict expression levels of each protein in the consortia. See Supplementary Information Section 1 for details on design of the consortia and the mathematical model. (B) Three consortia (A, B, and C) were established with different initial densities of strains expressing CFP, GFP, mOrange, and mCherry (shown as percentage values, top panel). Predicted and measured fluorescence intensities (bottom panel). The diameter of circles is proportional to relative density of the strain in the consortia. R2 values for model vs experimental results are shown. (mean±SD, n=3). (C) Based on the design of consortia B, consortium W was built using a weak RBS controlling GFP expression (1-log fold lower strength than the original RBS), and consortium L was built using a low copy number plasmid controlling expression of mOrange (1-log fold lower compared to a high copy number plasmid) (See Supplementary Information Section 1 for details). Predicted fluorescence intensities (dotted circles) match the experimentally measured values (filled circles). The fluorescence intensity is proportional to the diameter of the circles. (D) The fluorescent proteins were co-purified from the consortia: A, B, and C with strong RBS controlling expression of GFP (top panels); Aw, Bw, and Cw with a weak RBS controlling expression of GFP (bottom panels). Fluorescence intensities of each protein in the eluted fraction are normalized to total protein content. Each row corresponds to one consortium. Each column corresponds to one fluorescent protein. (mean±SD, n=3).

FIG. 2. Design and optimization of the synthetic bacterial consortia. (A) TraMOS is produced using a single bacterial consortium that expresses all the TraM proteins. The expression levels of each protein in the consortia are controlled by transcription rates (through plasmid copy number), translation rates (through RBS sequence), and relative strain densities. (B) In vitro expression activity of a mixture of Control IET (obtained by individually purifying the 11 IET factors) and Control AAT from a commercial source (left), and a mixture of TraMOS IET and TraMOS AAT III (right). Plasmid DNA was either absent (−) or present (+) in the reaction. Control IET and AAT exhibits higher GFP expression levels than TraMOS IET and AAT. (mean±SD, n=3 technical replicates). (C) Expression activities of mixtures of three TraMOS IET and Control AAT. The Control IET generates higher GFP expression levels than the three TraMOS IET variants. (mean±SD, n=3 technical replicates). (D) Expression activities of mixtures of four TraMOS AAT and TraMOS IET IV. TraMOS IET IV and AAT VI generates the highest GFP expression level when compared to the Control AAT. (mean±SD, n=3 technical replicates). (E) In vitro expression assay using TraMOS prepared from 34-strain consortia A and B. TraMOS B generates slightly lower GFP expression levels when compared to the control (93.7% of the activity in the control). (mean±SD, n=3 technical replicates). Means are significantly different by one-way ANOVA (P<0.0001) in (B) to (E). (F) Protein content of 34-strain TraMOS B in (E). (mean±SD, n=3). IET and AAT proteins represent more than 89% of the total protein content.

FIG. 3. Reducing number of bacterial strains in the synthetic consortia. (A) Design of the reduced-strain consortia. We constructed strains expressing either two TraM (2Tg strains) or three TraM genes (3Tg strains). All strains carry three plasmids, but 1Tg strains carry two unmodified plasmids (gray circles) and 2Tg strains carry one unmodified plasmid. For the 18-strain consortia, we supplemented the 17 2Tg strains with one 1Tg strain (expressing EF-G). For the 15 strain consortia, 11 3Tg IET or AAT strains were supplemented with three 2Tg strains and one 1Tg strain. See the detailed design of the consortia in Supplementary Information Sections 3.2 and 3.3. (B) In vitro expression of GFP using TraMOS. 18-strain TraMOS generates the highest expression level of GFP. Fluorescence intensities are normalized using the control (mean±SEM, n=3). Letters represent statistically different means by one-way ANOVA followed by Tukey's post test (P<0.01). (C) Quantification of TraM proteins in TraMOS from 34-(box), 18-(circle), and 15-strain (diamond) consortia. IET (left panel) and AAT (right panel) proteins are shown. Within each design of consortia, the quantified protein values are consistent across replicates (mean±SD, n=3). (D) Purity of 18- and 15-strain TraMOS from mass spectrometry quantification values. Percentages of normalized counts for IET and AAT factors, ribosomal proteins, and non-TraM proteins are shown (mean±SD, n=3). The results demonstrate high purity (>87%) of the TraM proteins. (E) In vitro expression of mCherry using TraMOS from 18-(white bars) and 15-strain (black bars) consortia. Fluorescence intensities are normalized using mean value within 18- or 15-strain consortia (mean±SD, n=3). The expression activities across replicates of consortia are not statistically different (one-way ANOVA, P values shown). The coefficient of variation (CV) is less than 7.1% for both designs of TraMOS, suggesting high reproducibility of the approach.

FIG. 4. Applications of the translation-mix one shot (TraMOS) in cell-free synthetic biology. (A) Four constructs with different translational regulatory sequences (Ngo 1, Ngo 1RBS, Ngo 7 and Ngo 7RBS) were tested using WCE (black bars) or 18-strain TraMOS (white bars). GFP expression intensities (normalized to the control without plasmids) of Ngo1 are the highest among the constructs. The letter above each bar represents groups with different means calculated by ANOVA (P<0.001) and Tukey post-test (P<0.01). (mean±SD, n=3). (B) A strategy to measure inhibitory function of chagasin protease inhibitors. Incubation of papain (Cys-protease) with its substrate FITC-Casein releases FITC, which fluoresces in solution. An inhibitor, generated in situ by TraMOS, reduces the protease activity of papain, reducing the free FITC levels. (mean±SD, n=3). (C) In vitro translation reaction using either WCE (top) or TraMOS (bottom) to express mCherry (gray bars) or WT chagasin (black bars), followed by the addition of FITC-casein, Papain, or both. TraMOS gives rise to less background FITC levels likely due to the absence of bacterial proteases. FITC fluorescence intensities are normalized to the FITC-casein control without papain (mean±SD, n=3). (D) 57 plasmids from a randomized library of chagasin mutants were analyzed in 384-plates (see Supplementary Information Section 4 for details). Normalized fluorescence intensity at 2 h is plotted for each of the variants (each replicate represented by a grey diamond) and for WT chagasin (black diamonds, in the first column). The gray shaded area represents the standard deviation of the FITC levels of the WT chagasin. The arrows indicate chagasin variants with consistent lower FITC intensities, hence higher inhibitory power on papain (white diamonds).

FIG. 5: Analysis of the fluorescent-protein consortia. (A) Predicted protein expression in fluorescent-protein consortia A, B and C, as a function of increasing relative densities of mOrange- (x-axis) and mCherry- (y-axis) expressing strains. Color gradient on the filled arrows represents relative density of each strain in the consortia from lowest (white) to highest (color). Increase in relative densities of strains expressing CFP and GFP is shown with the diagonal arrows. Each panel represents one fluorescent protein, and the diameter of the circle is proportional to the predicted fluorescent intensity on each consortia. (B) Correlation between fluorescence expression in consortia measured experimentally and fluorescence in elution fraction (normalized to maximal expression across consortia) in consortia A, B and C, shown as R² values. Circle diameter represents relative density of the strain in consortia. (mean±SD, n=3). (C) Correlation between predicted values from mathematical model and fluorescence intensities in elution fraction (normalized to maximal expression across consortia) of consortia A, B and C, shown as le values. Circle diameter represents relative density of the strain in consortia. (mean±SD, n=3).

FIG. 6: The impact of translation rates and gene-copy-number on protein yields. (A) Maps of plasmids used for fluorescent protein consortia. Plasmid pET15b (high copy number) was used to clone the four C-end 6x-His-tagged fluorescent proteins (C.FP), including GFP with both strong and weak RBS. pIURKL plasmid (low copy number) was used to express mOrange in consortium L (FIG. 1C). (B) Comparison of GFP expression using GFP_{weak} RBS, whose TIR is predicted to be 8.45 times lower than GFP_{strong} RBS. The results are confirmed by expression in vivo. (mean±SD, n=3). (C) Comparison of mOrange expression coded in high or low copy number plasmid in vivo. (mean±SD, n=3).

FIG. 7: Plasmid map of genetic constructs. Maps of plasmids created for the cloning of the TraM genes. pIURAH, pIURCM and pIURKL were derived from pET15b, pLysS and pSC101 respectively. The table shows the key features of the plasmid backbones, all of them conserved in the final pIUR plasmids.

FIG. 8. Optimization and development of functional 34-strain TraMOS. The Fig. shows the strategies used to optimize 34-strain TraMOS. a-e) the parameters considered for the design and optimization of the consortia are shown in gray boxes. Strain densities, plasmid copy number, and translation initiation rate (TIR) are considered for every steps, but shown only in TraMOS I (a). We used 1Tg strains coding for one TraM gene in all consortia, in either high or low plasmid copy number. “Activity” represents relative in vitro translation activity: −represents no activity, +/++ represents medium/high activity.

FIG. 9: Measurement of AAT activities in vitro. Determination of AAT activities from TraMOS AAT II subconsortia. The subconsortia were supplemented with each corresponding amino acid to determine the activity of each enzyme in the subconsortia. The negative control was not supplemented with amino acids. The level of released Pi is proportional to the AAT activity, and data is shown normalized to time=0. ns indicates results that are not significantly different from the control. *** represents significant difference, t-test P<0.001. (mean±SD, n=3).

FIG. 10. Impact of mass ratio IET:AAT on in vitro translation assays. (A) In vitro translation experiments combining different mass ratios (ng to ng of protein) of TraMOS IET IV to the Control AAT. Higher relative IET concentration increased the in vitro expression levels of GFP. (mean±SD, n=3). (B) In vitro expression activities of TraMOS IET IV:TraMOS AAT III mixtures at different mass ratios. Mass ratio of 14 gave rise to the highest expression level. (mean±SD, n=3).

FIG. 11. Optimization of TraMOS built with 2Tg and 3Tg strains. (A) The original 17-strain TraMOS, assembled only with 2Tg strains, presented no mCherry in vitro expression activity (−, first column). Supplementation with TraMOS IET IV mixture recovered the expression activity. Furthermore, addition of pure EF-G restored expression activity of the 17-strain TraMOS. Supplementing the 17-strain TraMOS with other elongation factors (individually), initiation factors (added individually or together) or all termination factors did not restore activity. (mean±SD, n=3). (B) In vitro expression of mCherry using two 18-strain TraMOS (18-strain TraMOS A and B) or two modified 17-strain TraMOS (17-TraMOS C and D) (Supplementary Information Section 3.2), with or without supplementation of IET TraMOS IV. Mixture IET TraMOS IV:AAT TraMOS VI is used as the control. 2Tg TraMOS B resulted in the highest expression activity. Based on these results, this consortium was selected for further experiments, and renamed as the 18-strain TraMOS. (mean±SD, n=3). (C) Two 15-strain TraMOS consortia were assembled as described (Supplementary Information Section 3.3). Strains expressing IET factors were present at higher relative densities in 15-strain B. Both 15-strain TraMOS A and B presented expression activities, although the activities in 15-strain TraMOS A were lower than TraMOS B. Expression activities were increased by the supplementation of TraMOS IET IV, but decreased by the supplementation of TraMOS AAT VI (probably due to dilution of IET factors). Activities of 15-strain B were the same for all conditions (15-strain TraMOS B was termed the 15-strain TraMOS hereafter). mCherry fluorescence intensities were normalized to the negative control without plasmid. (mean±SD, n=3).

FIG. 12. Western blot of strain 3Tg AAT 8. Strain 3Tg AAT 8 was induced with 0.5 mM IPTG for 5 hrs. The expressed proteins were purified as described in Methods. The purified fraction was subjected to western blot to identify His-tagged proteins. Both the total protein staining with Ponceau Red (P) and western blot with anti-His antibody (WB) are shown. We identified both thrS-N (blue arrow, 74.9 kDa) and cysS-C (green arrow, 53.2 kDa), but glyS (black arrow, 77.8 kDa) was not detected.

FIG. 13. Growth rates of the 1Tg, 2Tg, and 3Tg strains following induction of protein expression. Growth rates (GR) are calculated in absence (gray circle, uninduced) or presence (white circle, induced) of 0.5 mM IPTG. BL21(DE3) strain carrying the three empty pIUR plasmids is used as control (−, first column). (mean±SD, n=3). Impact of induction is calculated using the function (GR_{Induced}/GR_{Uninduced})*100. The table (bottom) shows the % GR_{Induced}/GR_{Uninduced} for IET, AAT and all (Total) strains (mean±SD, n=3). For example, the 3Tg IET strains exhibit overall lower growth rates after induction of gene expression (39% drop in average). The 2Tg IET and 1Tg IET strains exhibit 57% and 79% drop in growth rates after induction. This result confirms that growth rates of 3Tg strains are affected more by gene expression than growth rates of the 2Tg and 1Tg strains.

FIG. 14. Design of chagasin variants for in vitro screening. (A) Partial view of the crystal structure⁷ of Cys-protease (bottom, gray structure) and PbIP-C, a Cys-protease inhibitor from Plasmodium berghei (colored), showing their interacting surfaces. The backbone of interacting loops BC, DE and FG are shown in red, orange and yellow, respectively. The image was generated from PDB structure 3PNR, using Jmol software¹¹. (B) Multiple sequence alignments of the three loops BC, DE, and FG in the chagasin inhibitor family, which are responsible for direct interaction between the inhibitor and protease. The results show high degree of sequence conservation across members of this family. Triangles show the amino acids in these loops that i) are involved in direct interaction with the protease and ii) exhibit variations (i.e. not 100% conserved) among sequences (position 31 in loop BC; positions 64, 65 and 67 in loop DE; positions 91, 92, 93 and 99 in loop FG). Details of the sequences are shown in Table S10. First row (CAC39242) corresponds to chagasin from Trypanozoma cruzi, while the last row corresponds to PbICP-C (3PNR_B). (C) The variable positions are targeted for design of chagasin variants. We determine the potential variants accepted in those positions (based on the multiple sequence alignment), and design degenerated codons to introduce the mutations (Table S1. Expected frequency of each amino acid at each position is shown in the Fig. For example, the codon coding for L64 in loop DE will be targeted for mutation using the degenerated codon VKG, which code for a total of 6 codons: one for glycine (G), one for leucine (L), one for methionine (M), one for valine (V, 16.7% each) and two for arginine (R, 33%). The amino acid coded in the WT chagasin is shown in red. Considering all the potential combinations, a total of more than 160,000 variants could be generated with the strategy. (D) 24 clones from the chagasin mutant library were randomly selected and sequenced. The sequences are aligned as the predicted peptides coded by each clone. A high variability among sequences is observed, with modifications focused in the target positions. For example, the position 64 presents variability with L (WT), R, M, G and V, as expected.

FIG. 15. Expression of Chagasin in vitro and in vivo. Expression of WT Chagasin coded in WTCHGSN-pET15b plasmid. In vivo expression was conducted using different clones of the plasmid transformed into BL21(DE3) bacteria (left). In vitro expression was conducted using TraMOS and three different ribosomes concentrations (right). Images show western blots using the anti-flag monoclonal antibody. Molecular weight of chagasin is 13.1 kDa.

FIG. 16. Kinetic assay of FITC-casein proteolysis in 384-well plate. In vitro translation reactions were established using plasmid coding for mCherry (circles) or WT chagasin (squares) and incubated for 3 h at 37° C. Next, we added FITC-casein without (empty markers) or with (filled markers) papain, and FITC fluorescence was read for 2 h. FITC-fluorescence intensities are normalized to the value at t=0. The shaded backgrounds represent the SD. (mean±SD, n=3).

FIG. 17. Mathematical model to predict protein output in TraMOS. (A) Low stochastic variation between biological replicates. The protein yield for the three biological replicates of the 34-(left) and 18-(right) strain consortia are correlated pairwise using a Pearson correlation coefficient (log scale). (B) Predictive results from mathematical models vs proteomic data. Both axis are shown in log scale. (mean±SEM, n=3 for measured values). Results from the 34-strain consortia are used to estimate the synthesis rate of each protein and therefore has a perfect correlation (r=1). The obtained parameters are then used to model the 18-strain consortia. For both consortia, Nᵢ(0) is equal to the relative cell density times 0.01 (to model the OD600 of an initial inoculum). r is calculated for each strain and K=0.8. Cᵢ equals 10 for high copy number and 1 for low copy number plasmids. The length of the gene, lᵢ, is determined for each gene and Δ=1. See Supplementary Info Section 5 for details of the model.

### DETAILED DESCRIPTION

The present invention provides a new approach to produce a desired multi-protein complex (e.g., one useful for in vitro translation of mRNAs or TraM) by exploiting microbial consortia (i.e., associations of multiple strains of microorganisms living in a single culture). The invention is based on the design principle of distributing metabolic burden from protein synthesis across multiple microbial strains. Different bacterial strains are engineered to express distinct proteins in a single culture (referred to as TraM one shot or TraMOS). Subsequently, all the proteins are purified using a single affinity chromatography step.

As explained in detail below, the relative amount of each protein in the complex is regulated such that the complex efficiently produces the desired final product (e.g., a translated polypeptide in the case of TraMos).

The proteins of the invention can be made using standard methods well known to those of skill in the art. Recombinant expression in a variety of microbial host cells, including E. coli, or other prokaryotic hosts is well known in the art.

Polynucleotides encoding the desired proteins in the complex, recombinant expression vectors, and host cells containing the recombinant expression vectors, as well as methods of making such vectors and host cells by recombinant methods are well known to those of skill in the art.

The polynucleotides may be synthesized or prepared by techniques well known in the art. Nucleotide sequences encoding the desired proteins may be synthesized, and/or cloned, and expressed according to techniques well known to those of ordinary skill in the art. In some embodiments, the polynucleotide sequences will be codon optimized for a particular recipient using standard methodologies. For example, a DNA construct encoding a protein can be codon optimized for expression in microbial hosts, e.g., bacteria.

Examples of useful bacteria include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. The nucleic acid encoding the desired protein is operably linked to appropriate expression control sequences for each host. For E. coli this includes a promoter such as the T7, trp, or lambda promoters, a ribosome binding site and preferably a transcription termination signal. The proteins may also be expressed in other cells, such as mammalian, insect, plant, or yeast cells.

Once expressed, the recombinant proteins can be purified according to standard procedures of the art, including ammonium sulfate precipitation, affinity columns, column chromatography, gel electrophoresis and the like. In a typical embodiment, the recombinantly produced proteins are expressed as a fusion protein that has a “tag” at one end which facilitates purification of the proteins. Suitable tags include affinity tags such as a polyhistidine tag which will bind to metal ions such as nickel or cobalt ions. Other suitable tags are known to those of skill in the art, and include, for example, epitope tags. Epitope tags are generally incorporated into recombinantly expressed proteins to enable the use of a readily available antibody to detect or isolate the protein.

### EXAMPLES

The following examples are offered to illustrate, but not to limit the claimed invention.

### Methods

E. coli Strain and Plasmids

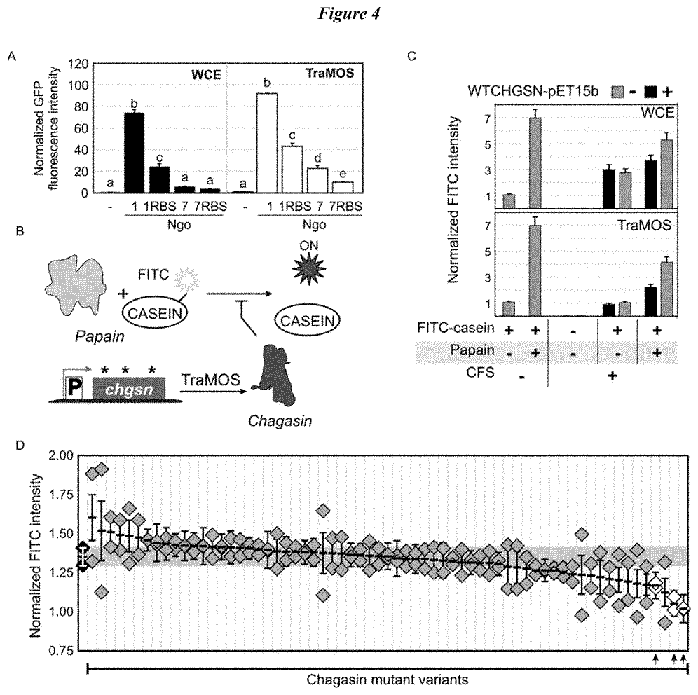

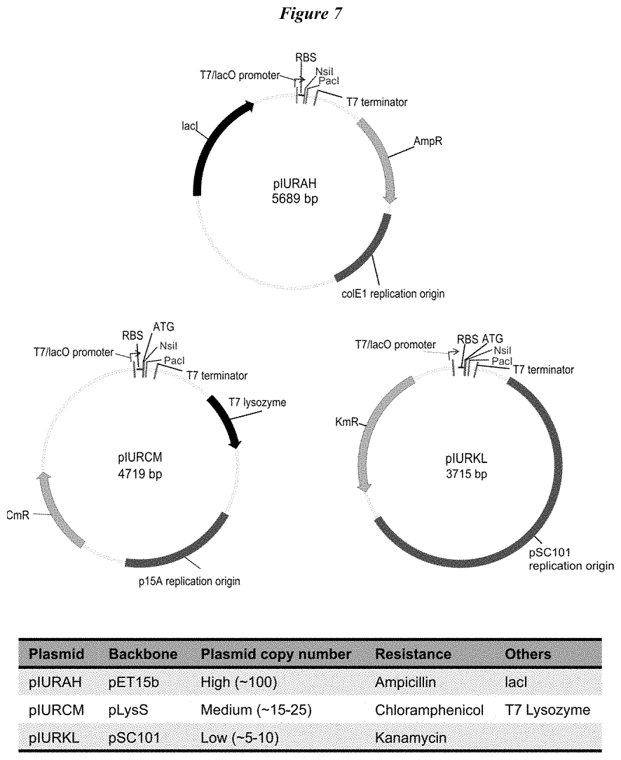

E. coli BL21 (DE3)-pLysS strain was used to construct the consortia that express fluorescent proteins. BL21 (DE3) was used to construct the consortia that express TraM proteins. Genomic DNA from E. coli MG1655 was prepared using Wizard Genomic DNA Purification Kit (Promega). pET15b (Novagen), pLysS (Novagen), and pSC101 (Manen and Caro, 1991) plasmids were used to create new plasmids pIURAH, pIURCM and pIURKL, respectively (Supplementary Information Section 2 for details). The three plasmids carry an NsiI/PacI cloning site downstream of a PT7/lacO hybrid promoter. pIURAH contains the Amp^{R}/ColE1 replication origin and expresses lad, pIURCM contains the Cm^{R}/p15A replication origin and expresses T7 lysozyme, and pIURKL contains Km^{R}/pSC101 replication origin (FIG. 7). All primers used in the work are listed in Table S1. The construction of WTCHGSN-pET15b and its variants is described in details in Supplementary Information Section 4. Accession numbers for Ngo plasmid series used in FIG. 4A are: Ngo1 KX787434, Ngo1RBS KX787435, Ngo7 KX787436, Ngo7RBS KX787437).

### Cloning of Fluorescent Proteins

CFP, GFP, mOrange and mCherry genes were amplified with the insertion of a 6x-His tag sequence in the C-end using specific primers. The amplicons were cloned into XbaI/NcoI-digested pET15b plasmid using Gibson Assembly (New England Biolabs), yielding C.CFP-, C.GFP, C.mOrange- and C.mCherry-pET15b plasmids. mOrange was cloned into NsiI/PacI-digested pIURKL using Gibson Assembly (yielding C.mOrange-pIURKL). C.GFP-pET15b RBS sequence was modified by digesting the plasmid XbaI/NcoI and inserting a PCR product (generated using primers that introduced a weaker RBS) by Gibson Assembly, to produce C.GFP_{weak}-pET15b.

Analysis of the Consortia that Express Fluorescent Proteins

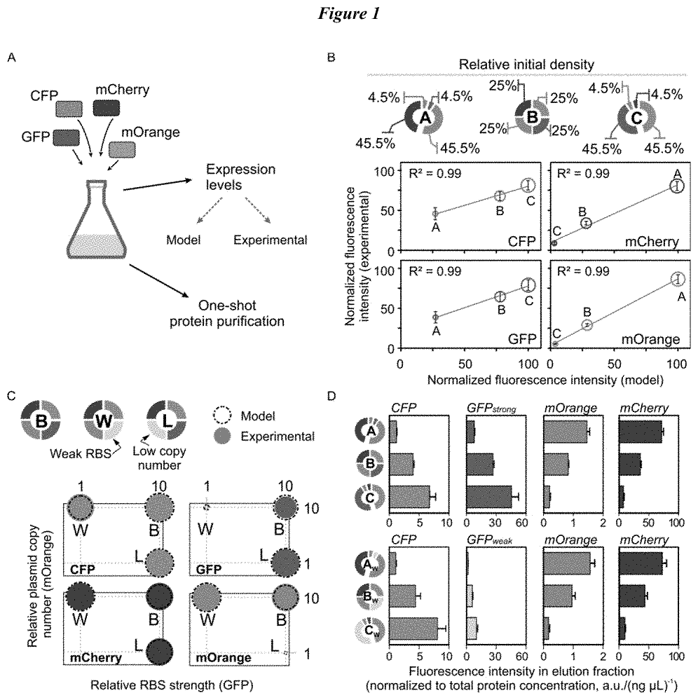

The plasmids expressing each fluorescent proteins (C.CFP-, C.GFP, C.GFPweak-, C.mOrange- and C.mCherry-pET15b) were independently transformed into BL21 (DE3)-pLysS. The resulting strains were Amp^{R}/Cm^{R}. C.CFP-, C.GFP, and C.mCherry-pET15b plasmids were co-transformed with the unmodified pIURKL in BL21 (DE3)-pLysS. C.mOrange-pIURKL was co-transformed with the unmodified pET15b into BL21 (DE3)-pLysS cells. These strains (Amp^{R}/Cm^{R}/Km^{R}) were used to construct consortium L (FIG. 1C). To establish the consortia, all the strains were grown overnight with antibiotics at 37° C. (in all cases, carbenicillin was used instead of ampicillin). The overnight cultures were premixed at specific relative densities and inoculated 1/200 in M9 media supplemented with 0.1% casamino acids, 0.1% glucose, and antibiotics. Culture volume was 200 μL in 96-well plates. Plates, covered with plastic lid, were incubated for 1 hr at 37° C. with shaking cycles of 20 sec ON, 40 sec OFF in an m1000Pro Infinite reader (Tecan). Then, cultures were induced with 1 mM IPTG and measured for 16 hrs. OD600 and fluorescence for each protein were recorded at every 15 min. Fluorescence intensity/OD600 was calculated for each time point.

### One-Shot Protein Purification From the Consortia That Express Fluorescent Proteins

Premixed consortia were inoculated in triplicates at 1/250 dilution in 5 mL M9 media supplemented with 0.1% casamino acids, 0.1% glucose, and carbenicillin/chloramphenicol. After 2 hrs, cultures were induced with 1 mM IPTG for 6 hrs. Cells were collected and lysed in CelLytic B Buffer (Sigma Aldrich) supplemented with Benzonase (Novagen) 0.02% v/v. Cell debris was removed by centrifugation (20,000g for 15 min at 4° C.) and supernatant was stored for purification. The supernatant was applied to 100 μL of Ni-NTA resin (Life Technologies) previously equilibrated with a binding buffer (50 mM Tris-HCl pH 7.5, 100 mM NaCl and 30 mM Imidazole). The resin was washed with 1 mL of wash buffer (binding buffer supplemented with 1% Tween 20) and 1 mL of binding buffer. Proteins were eluted in elution buffer (50 mM Tris-HCl pH 7.5, 100 mM NaCl and 250 mM Imidazole). Total protein concentration was quantified using 660 nm Protein Assay (Thermo Scientific). Fluorescence intensities of CFP, GFP, mOrange, and mCherry were determined using NanoQuant plate (Tecan) and m1000Pro Infinite reader.

### Cloning of TraM Genes

The 34 TraM genes (Table S2) were cloned from E. coli MG1655 genomic DNA, using specific primers to introduce either N- or C-end 6x His tag, as well as NsiI and PacI restriction sites. The genes were amplified by PCR. C-end tagged TraM genes were reamplified using the proper forward primer and a universal reverse primer (TramCend_Cloner). All fragments were cloned using Gibson Assembly (New England Biolabs) into pIUR plasmids, which were digested by NsiI and PacI. All TraM genes were amplified using one set of primers except asnS-N (1425 bp), which was amplified using a primer set for base pairs 1-742 and another primer set for base pairs 716-1425. These two fragments were fused together in Gibson Assembly reactions to clone the full length gene in pIUR plasmids. All positive clones were confirmed by DNA sequencing and western blots to confirm identity of the proteins expressed in BL21 (DE3) induced with IPTG.

### Creation of 1Tg, 2Tg and 3Tg TraMOS Strains

1Tg strains were created by simultaneous transformation of pIURAH or pIURKL genes coding for a single TraM genes, plus unmodified pIURCM and pIURKL or pIURAH, accordingly, into BL21 (DE3) competent cells (Table S3). 2Tg strains were generated by co-transformation of pIURAH and pIURKL plasmids coding for TraM genes, plus unmodified pIURCM. Finally, 3Tg strains were created by co-transforming the three pIUR plasmid coding for TraM genes. All strains were confirmed by expression of the target proteins, which were analyzed by western blot using anti-His antibody. All strains were selected in LB-agar plates supplemented with the three antibiotics and stored as glycerol stocks.

### Growth Rate Calculation

In order to determine growth rate of the 1Tg, 2Tg, and 3Tg strains, we first grew the strains overnight at 37° C. in LB supplemented with antibiotics. The overnight cultures were inoculated at 1/200 dilution into 96-well plates containing 200 μL of LB with antibiotics. The plate, covered with plastic lid, was incubated for 1 hr at 37° C. with shaking cycles of 20 sec ON, 40 sec OFF in plate reader, and water or IPTG (0.5 mM final concentration) was added. OD600 was registered over 8 hrs. Growth rates were calculated using the program GrowthRates (Hall et al., 2014).

### Buffers Used for Purification of TraMOS Proteins

Buffers for purification of TraMOS proteins were prepared following previous work (Shimizu and Ueda, 2010), with slight modifications. Buffer A: 50 mM HEPES pH 7.5, 1 M Ammonium chloride, 10 mM Magnesium chloride; Buffer B: 50 mM HEPES pH 7.5, 500 mM Imidazole, 10 mM Magnesium chloride; Buffer HT: 50 mM HEPES pH 7.5, 100 mM potassium chloride, 10 mM Magnesium chloride, 7 mM 2-mercaptoethanol; Buffer HT+: 50 mM HEPES pH 7.5, 100 mM potassium chloride, 50 mM potassium glutamate, 10 mM Magnesium chloride, 7 mM 2-mercaptoethanol. 2-mercaptoethanol was freshly prepared before use in all cases.

### Preparation of the Control IET

The 1Tg strains coding for the 11 initiation, elongation, and termination factors were grown overnight at 37° C. in 3 mL of LB media supplemented with carbenicillin/chloramphenicol/kanamycin. Each strain was individually inoculated in a flask containing 600 mL LB with antibiotics at 1/250 dilution, and grown for 90 min at 37° C. before induction with 0.5 mM IPTG for 4 hrs. Cells were collected by centrifugation and stored at −80° C. overnight. Next day, cell pellet was resuspended in 5 mL per g of cells in a binding buffer (Buffer A:Buffer B 97.5:2.5 with 7 mM 2-mercaptoethanol). Cells were lysed by sonication and cell debris was removed by centrifugation (20,000g, 15 min, 4° C.). Supernatant was applied to a 1 mL HisTrap FF column (GE Healthcare Life Sciences) previously equilibrated with 10 volumes of the binding buffer. Each column was washed with 10 volumes of the binding buffer and 10 volumes of a wash buffer (Buffer A:Buffer B 95:5 plus 7 mM 2-mercaptoethanol), and then eluted with 7 mL of an elution buffer (Buffer A:Buffer B 20:80 plus 7 mM 2-mercaptoethanol). Each elution fraction was dialyzed for 6 hrs against Buffer HT, followed by overnight dialysis against Buffer HT supplemented with glycerol 20%. Proteins were then concentrated by ultrafiltration using Amicon Ultra-4 Centrifugal Filter Units 3,000 MWCO (EMD Millipore). Protein concentrations of each factor were analyzed using the 660 nm Protein Assay. Control IET was prepared by combining all the factors at the concentrations shown in Table S6. Control AAT is a mixture of all the tRNA-amino acyl transferases from E. coli (Sigma Aldrich).

### Establishment, Induction, and Purification of the TraMOS Consortia

Each strain required to establish a consortium was grown overnight from glycerol stocks in LB media supplemented with the antibiotics at 37° C. Details on the design of the strains and establishment of consortia are described in Supplementary Information, Section 3. The overnight cultures were used to establish consortia by mixing the strains at the indicated ratios (ratio represent % of the strain in the total volume of the mix). The consortia were then inoculated 1/500 into 600 mL LB with antibiotics and grown 90 minutes before induction for 4 hrs with 0.5 mM IPTG, except the 15-strain consortia that were inoculated 1/200, grown 90 minutes and induced for 5 hrs with 0.5 mM IPTG. TraM proteins from the cultures were purified as described above, with the exception that the final overnight dialysis step was performed against Buffer HT+. Protein identification and quantification were performed by the Proteomics Core Facility, Genome Center at University of California, Davis. Samples were digested with trypsin, and peptides were analyzed using Q-Exactive liquid chromatography tandem mass spectrometry (LC-MS/MS). Results were analyzed using X!tandem against a customized database that includes the total BL21 (DE3) and the 6x-His-tagged TraM proteins.

### SDS-PAGE and Western Blot

Proteins were separated by SDS-PAGE using 8-16% Mini-PROTEAN TGX precasted gels (Bio-Rad). For western blot, proteins were transferred to nitrocellulose membranes using Trans-Blot Turbo Transfer System (Bio-Rad). For the quantification of total protein amount, gels were stained using Coomassie Brilliant Blue Electrophoresis Gel Stain (G-Biosciences). Nitrocellulose membranes were stained using Ponceau-S Membrane Stain (G-Biosciences), imaged and subsequently blocked with 5% Dry fat milk in TBS-T buffer (TBS plus 0.1% Tween-20). Membranes were exposed to either Mouse Anti-6x-His Epitope Tag HIS.H8 or Rat Anti-FLAG Epitope Tag L5 to detect His-tagged or FLAG-tagged proteins, respectively. Following washes with TBS-T plus 0.1% BSA, membranes were exposed to HRP-conjugated secondary antibodies Goat anti-Mouse IgG or Goat anti-Rat IgG for His-tagged or FLAG-tagged proteins, respectively. Membranes were developed using Clarity Western ECL Substrate (Bio-Rad). Gels and membranes were imaged using a PXi Imaging system (Syngene).

### Preparation of S12 Whole Cell Extract (WCE)

Overnight cultures of BL21 (DE3) strain were diluted 1:1000 in fresh LB containing 0.4 mM IPTG. Bacteria were collected and washed twice with PBS (4,000×g, 10 min, 4° C.) after growing at 30° C. for 6 h. The bacterial pellet was resuspended in sonication buffer (10 mM Tris-acetate pH 7.6, 14 mM Magnesium acetate, 60 mM Potassium gluconate, 1 mM DTT) to a final concentration of 1 g/mL. The resuspended bacteria were lysed by sonication. Cell lysates were centrifuged at 12,000×g for 20 min at 4° C. The supernatant was incubated at 37° C. for 30 minutes. The resulting WCE was aliquoted and stored at −80° C.

### In Vitro Translation

2× reaction buffer contained amino acid mix 110 mM (each amino acid 5.4 mM), tRNA (Roche) 108 U_{A260}/mL, ATP 7.5 mM, GTP 5 mM, CTP 2.5 mM, UTP 2.5 mM, Creatine phosphate 100 mM, Folinic acid 60 μg/mL, HEPES-KOH 7.6 100 mM, Potassium glutamate 700 mM, Magnesium Acetate 36 mM, Spermidine 2 mM, DTT 10 mM, BSA 1 mg/mL, Creatine Kinase (Roche) 162 μg/ml, Myokinase (Sigma Aldrich) 100 μg/mL, Diphosphonucleotide Kinase (Sigma Aldrich) 8.16 μg/mL, T7 RNAP (New England Biolabs) 400 U/μl, RNAse inhibitor (New England Biolabs) 0.8 U/μl. Amino acid mixture was prepared as described in a previous work (Caschera and Noireaux, 2015). Reactions (final volume 5 μL) were established by combining 2× reaction buffer, cell-free systems, 1.3 μM ribosomes (New England Biolabs), and 2-5 ng of plasmid DNA. When reactions were conducted using the S12 WCE, T7 RNAP was not included in the 2× reaction buffer, and ribosomes were not added. After mixing, reactions were incubated 4 h at 37° C., and measured using the NanoQuant plate as described above.

### Papain Inhibition by Chagasin Variants In Vitro

In vitro transcription/translation reactions (final volume 5 μL) were performed using either C.mCherry-pET15b or WTCHGSN-pET15b plasmids (Supplementary Information Section 4) and incubated for 3 hrs at 37° C. Next, the reactions were supplemented with 2 μl of PBS and either 1 μl of FITC-Casein (AnaSpec)+1 μl of PBS or 1 μl of FITC-Casein+1 μl of papain (Sigma Aldrich). Final concentration of FITC-Casein was 0.04 μg/μL. Final concentration of papain was 0.4 ng/μL. Each reaction was allowed to proceed for 2 hrs at 37° C. and measured for FITC fluorescence intensities using NanoQuant plate as described above. Data was normalized using the fluorescence intensities of the control (FITC-Casein in PBS without CFS). Reactions in 384-well plates were set up in a similar way, except that plates were covered with film and placed in the plate reader to measure FITC-fluorescence intensities using a 2 h kinetic cycle at 37° C. with measurement at every 5 min. Fluorescence data was normalized using the data at time=0. For the screening of the library (details in Supplementary Information Section 4), different plasmids were added to replicate wells.

### Mathematical Modeling and Statistical Analysis

Models' details are described in Supplementary Information, Sections 1 (fluorescent protein consortia) and 5 (TraMOS predictive model). Codes were written using MatLab. All statistical analysis were performed using GraphPad Prism 5.0 software.

### Results and Discussion

### Establish Synthetic Biological Approaches to Control the Synthesis of Multiple Proteins in Synthetic Bacterial Consortia

The preparation of multiprotein complexes requires a tight control over expression levels of each protein in the consortium, in order to match their working concentrations in the final product. For coarse-grained regulation of protein amount, the cell number of each bacterial strain is controlled through its relative density in the consortium. For fine-grained regulation of protein amount, transcription and translation levels are controlled using synthetic genetic constructs. To simplify the genetic constructs, we use a single regulatory circuit based on the PT7/lacO hybrid promoter to activate protein expression by T7 RNAP and inhibit it by LacI. In addition, the transcription rate is controlled using plasmids with different copy number, whereas the translation rate is modulated by altering the ribosomal binding site (RBS) sequence of the target gene.

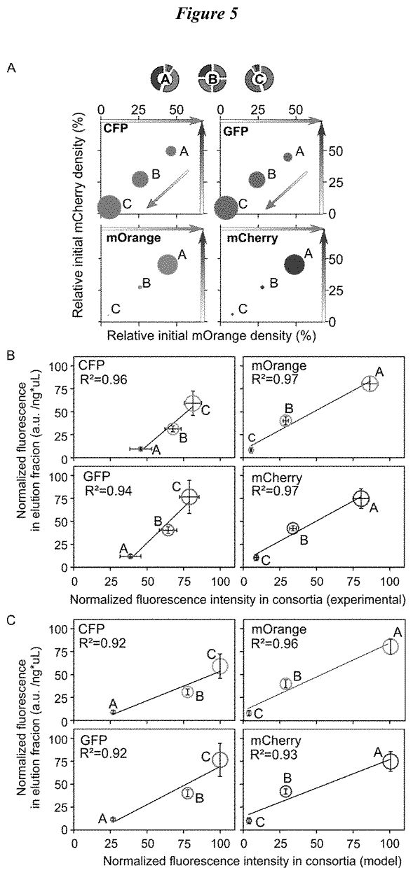

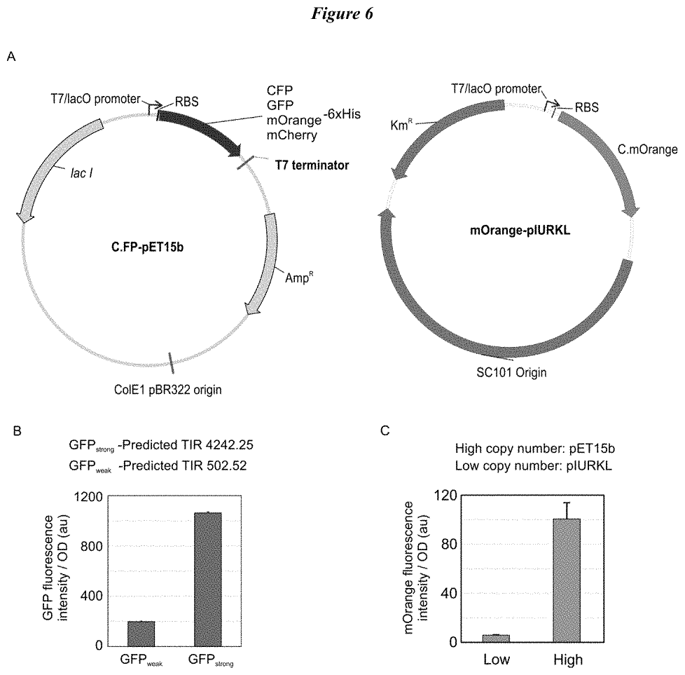

To define the control mechanisms, we designed consortia composed of four strains, each expressing one of four different fluorescent proteins CFP (cyan fluorescent protein), GFP (green fluorescent protein), mCherry and mOrange (FIG. 1A and Supplementary Information). We created a simple mathematical model with a system of ODE to calculate bacterial growth and expression level of each fluorescent protein in the consortia with parameters adjusted for initial bacterial density, plasmid copy number, and translation rates (Supplementary Information). Consistent with model predictions, we found that protein levels in the consortia were controlled by the relative initial density of each strain in the consortia (FIG. 1B and FIG. 5A), as well as the transcription and translation rates of the proteins (FIG. 1C and FIG. 6). We also confirmed that the four proteins were co-purified from the consortia at levels comparable to their expression levels in the consortia (FIG. 1D and FIGS. 5B and 5C).

The mathematical model suggested that protein levels in the consortia can be controlled by changing the relative density of each strain in the consortia (FIG. 1B) and by modifying transcription or translation rates of specific proteins (FIG. 1C). To validate the modeling predictions, we experimentally established consortia A, B, and C using four BL21(DE3)-pLysS strains, each transformed with a high copy number plasmid expressing a fluorescent protein tagged with a C-terminal 6x-Histag for immobilized metal affinity chromatography (IMAC) purification. Each strain was grown overnight and used to establish the consortia by mixing the strain at the indicated ratios (FIG. 1B). Consistent with modeling results, the total expression level of each protein changed proportionally to the initial relative density of each strain in the consortium. Through these experiments, we were able to control protein expression using relative strain densities in bacterial consortia.

### Design and Optimization of Bacterial Consortia to Produce Functional TraM in a Single Purification Step

Next, we extended the control mechanisms to produce multi-protein complexes, using TraM as a model multi-protein complex. To start, we designed three plasmids with compatible replication origins and distinct copy number, each carrying a hybrid PT7/lacO promoter, cloning sites, and T7 RNAP terminator sequence (FIG. 7). The 34-TraM genes (Table S2) were cloned into the three plasmids with a 6x His tag located at either the N- or C-end as previously reported (Shimizu and Ueda, 2010). BL21 (DE3) E. coli cells were co-transformed with the three plasmids that expressed either TraM genes or nothing, creating 34 strains expressing a single TraM gene (1Tg strains in Table S3). The RBS Calculator tool was used to estimate translation rates of each gene (Salis, 2011) (Table S2).

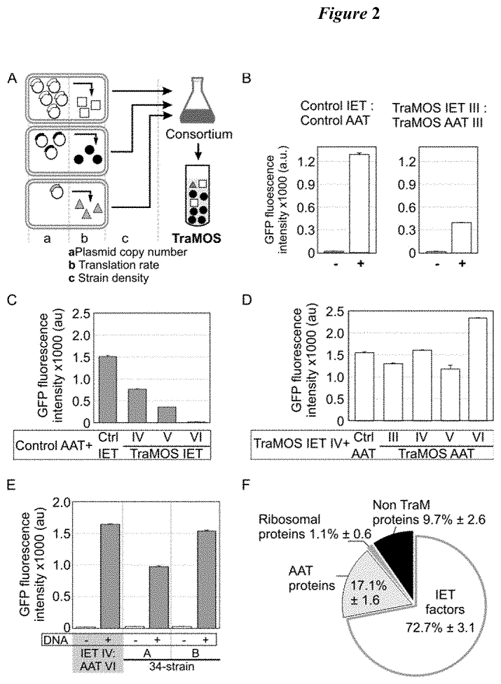

As initial attempts resulted in TraMOS with low expression activities (see Supplementary Information Section 3.1.1 for details), we reduced the complexity of the TraM system by creating sub-consortia based on common functions of the proteins: the IET consortium with 11 strains, each coding for one of the IET genes (Supplementary Information Section 3.1.2); and the AAT consortium with 23 strains, each expressing a single AAT gene (Supplementary Information Section 3.1.3). Based on reported concentrations of the proteins in an optimized system (Kazuta et al., 2014) (Table S4), we designed the consortia to achieve comparable expression levels of each TraM factor, taking into consideration the plasmid copy number, predicted translation rates, and relative densities of the strains (Tables S2 and S6). The established consortia were used to co-purify either the 11 IET (TraMOS IET III) or the 23 AAT (TraMOS AAT III) proteins from single bacterial co-cultures. In parallel, we prepared an IET mixture from individually purified IET proteins, termed Control IET. We then tested the GFP expression activity using the protein mixtures. Indeed, the TraMOS assembled from separate TraMOS IET III and TraMOS AAT III cultures gave rise to GFP expression (FIG. 2B), although the expression level was lower than that of the mixture assembled from Control IET and Control AAT (commercially available mixture of all the AAT factors). These results support the feasibility of producing TraM using synthetic bacterial consortia.

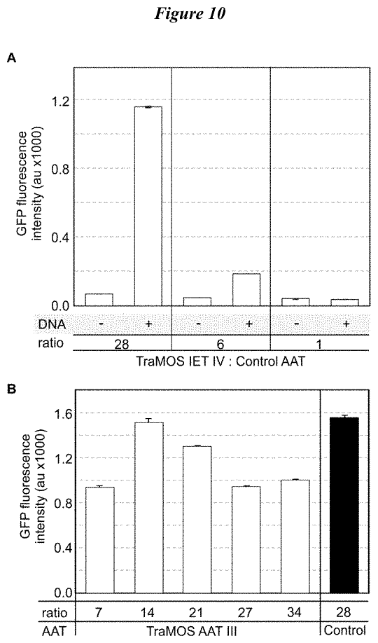

To further improve TraMOS IET, we created three additional IET consortia, termed IET IV, V and VI, in which the relative densities of bacterial strains were adjusted (Supplementary Information Section 3.1.2). When TraMOS IETs were combined with Control AAT (FIG. 2C), TraMOS IET IV presented half of the expression activity observed with Control IET, although its activity was higher than the activity of TraMOS IET III. Next, we constructed different TraMOS AAT consortia by adjusting relative densities of the strains and plasmid copy number for four of the AAT genes (Supplementary Information Section 3.1.3). We also found that expression activity of TraMOS was maximal when the mass ratio of IET:AAT was 14 (FIG. 10B), suggesting that IET factors might be limiting protein synthesis rates. Using an optimized IET:AAT ratio, we measured the expression activity of all the TraMOS AAT versions in combination with TraMOS IET IV. TraMOS AAT VI showed 50% higher expression activity when compared to the Control AAT (FIG. 2D). These results establish a strategy to group proteins based on either functions or pathways for assembling the final complete consortia.

The above divide-and-conquer strategy generated the necessary insights into setting up full TraMOS consortia A and B, each with 34 bacterial strains combined in a single culture. Overall IET strains density in TraMOS A was lower than that of TraMOS B (Supplementary Information Section 3.1.4). Both TraMOS exhibited expression activities (FIG. 2E), but TraMOS B presented higher activity than TraMOS A, and 93.6% of the activity of the IET IV:AAT VI mixture. Furthermore, mass spectrometry results of TraMOS B (hereafter referred to as 34-strain TraMOS) suggest a high degree of purity and reproducibility of the multi-protein complex using the synthetic bacterial consortia (FIG. 2F and Table S5). These results demonstrate successful purification of multi-protein complexes from a single consortium with the highest number of synthetic bacterial strains described to date.

Reproducible Preparation of TraMOS Using Bacterial Consortia with Reduced Strain Number

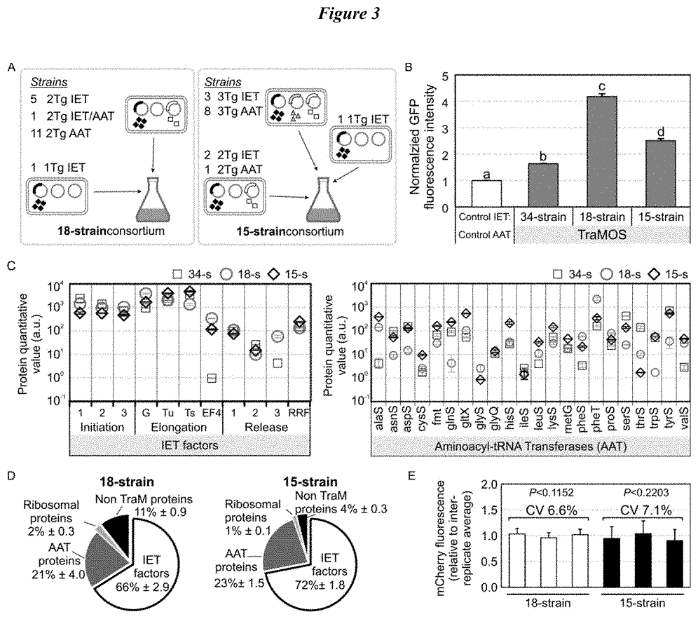

A microbial-consortia approach for purifying multi-protein complexes would be less susceptible to experimental errors if the consortia have lower number of bacterial strains. To this end, we first created 17 strains coding for two TraM genes (2Tg) and 11 strains expressing three TraM genes (3Tg) simultaneously (Table S3). Then, we used these strains to establish two new consortia (FIG. 3A): one 18-strain TraMOS consortium consists of the 17 2Tg strains supplemented with one 1Tg strain (Supplementary information Section 3.2 and Table S8); and a 15-strain TraMOS consortium with eleven 3Tg, three 2Tg and one 1Tg strains (Supplementary information Section 3.3 and Table S9). Both 18- and 15-strain TraMOS yielded higher activities when compared to Control IET:Control AAT (4.1- and 2.5-fold, respectively) and 34-strain TraMOS (FIG. 3B). The design of the reduced-strain consortia highlight the importance of the fine control of gene expression using both gene copy number and translation initiation rates.

Reproducibility is a critical, yet non-trivial aspect of multi-protein purification approach based on microbial-consortia. To this end, we produced TraMOS replicates from 18- and 15-strain consortia. Next, we identified and quantified the protein composition of the TraMOS using mass spectrometry (FIG. 3C), demonstrating that purity of 18- and 15-strain TraMOS is high (>87%, FIG. 3D). The 18- and 15-strain TraMOS also gave rise to consistent expression activities across independent replicates collected from independent experiments (coefficients of variation <7.1%). These results corroborate robustness of our approach to experimental variation (FIG. 3E). In addition, a deterministic mathematical model for 18-strain TraMOS (see Supplementary Information Section 5 and FIG. 17B) is formulated using data from the 34-strain consortia, and shows a correlation of r=0.65 with the experimentally observed protein yields. We note that this version of the model can be improved further by incorporating experimentally measured parameters. The model represents a step toward the mathematically-guided design of consortia for multiprotein complexes preparation in future work.

### Applying TraMOS in the Prototyping of Parts for Synthetic Biology Applications

By reducing the time and cost associated with preparing multi-protein complexes, our approach essentially enables high-throughput applications of TraMOS without investment into additional purification equipment. Here, we utilized TraMOS to test translation activity from a set of different plasmids expressing GFP with variable RBS sequences. It has been shown by biophysical modeling and experimental data that the sequence comprising 35 nucleotides up- and down-stream from the initiation codon affect the translation rate (Espah Borujeni et al., 2014; Mutalik et al., 2013). The RBS Calculator predicted that the translation rates of the four variants presented here are different. Using bacterial S12 whole cell extract (WCE) to test in vitro transcription/translation activity, we observed significant differences in expression activities of two variants (Ngo1 and Ngo1RBS) relative to the negative control (FIG. 4A). In contrast, TraMOS resulted in significantly different activities of all four promoter variants (FIG. 4A), likely due to a higher signal-to-background ratio of well-defined protein mixture.

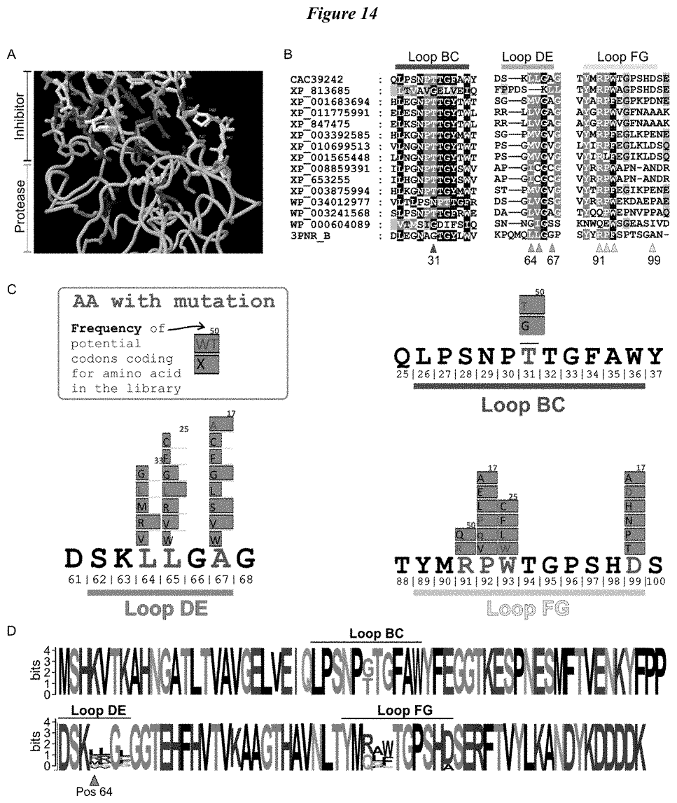

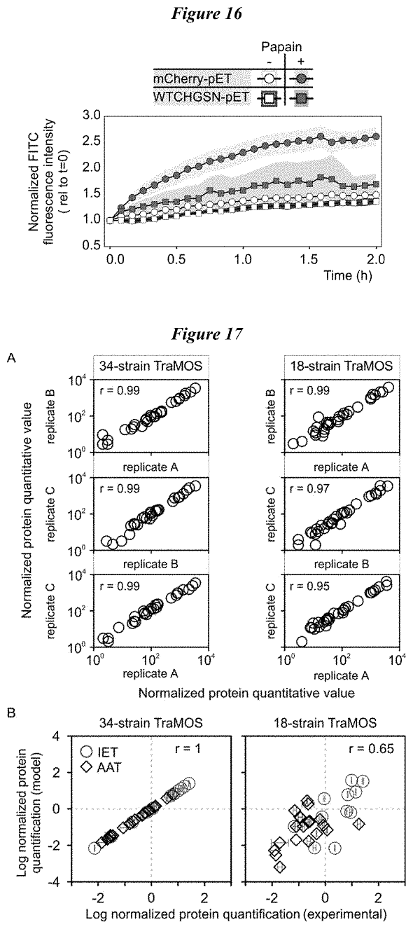

In addition, we demonstrate the utility of TraMOS by incorporating it into a screening assay of protease inhibitors. Cysteine proteases, important in parasite pathogenesis, are inhibited by a family of small peptides, including the Trypanozoma cruzi inhibitor chagasin (Redzynia et al., 2009). Chagasin binds to the protease, blocking its active site in three loops, BE, CD and FG (FIG. 14A) (Pandey, 2013). We created a library of mutants targeting amino acids in these loops (Supplementary Information Section 4, FIG. 14B and 14C). Next, we expressed these mutants using TraMOS to isolate variants with improved inhibition of a cysteine protease Papain (FIG. 4B). When wild type Chagasin was expressed using either WCE or TraMOS, it inhibited activities of Papain (FIG. 4C). WCE exhibited background protease activities, as shown by the fluorescence intensity without Papain that was higher than the basal level (FIG. 4C). Conversely, TraMOS did not show background protease activities, confirming its advantage in reducing protein impurities (proteases in this case) that cause background activities of cell-free protein assays (FIG. 4C). We also confirmed the anti-Papain activity of WT chagasin in kinetic assays using 384-well plate with 5 μL reaction volume (FIG. 16). Next, we screened 57 chagasin variants from our library and quantified their inhibitory activities using TraMOS. Comparing to WT chagasin (FIG. 4D, first column in black diamonds), we identified 3 variants that consistently presented higher inhibitory activities, with 15.7%, 28.3% and 32.6% increase respectively (FIG. 4D, white diamonds, denoted with arrows). Together, these assays support the feasibility of using TraMOS for high throughput screening assays.

Our work has wide impact on cell-free synthetic biology by enabling the production of pure translation machinery through a simple and fast method. The approach is compatible with the existing equipment of most labs that perform protein purification routinely, allowing easy implementation of TraMOS and democratizing access to this system for high-throughput cell-free applications. Furthermore, our work establishes a microbial-consortia based approach for the purification of multi-protein complexes, which may be generalized to the production of other systems, such as the 28-enzyme system for purine nucleotide synthesis (Schultheisz et al., 2008) and the seven-enzyme system for production of an anti-malaria artemisinin precursor, amorpha-4,11-diene (Chen et al., 2013). Application of our strategy to other multi-protein complexes will require further adjustment of purification conditions (buffer composition or alternative tags). Finally, to enable autonomous control of protein expression in synthetic bacteria consortia, we may incorporate inter-strain communication (Groβkopf and Soyer, 2014) that responds to quorum sensing signals or nutrients (Scott and Hasty, 2016).

### REFERENCES

Arai, T., Matsuoka, S., Cho, H. Y., Yukawa, H., Inui, M., Wong, S. L., and Doi, R. H. (2007). Synthesis of Clostridium cellulovorans minicellulosomes by intercellular complementation. Proceedings of the National Academy of Sciences of the United States of America 104, 1456-1460.

Brenner, K., You, L., and Arnold, F. H. (2008). Engineering microbial consortia: a new frontier in synthetic biology. Trends in biotechnology 26, 483-489.

Caschera, F., and Noireaux, V. (2015). Preparation of amino acid mixtures for cell-free expression systems. BioTechniques 58, 40-43.

Caschera, F., and Noireaux, V. (2016). Compartmentalization of an all-E. coli Cell-Free Expression System for the Construction of a Minimal Cell. Artificial life 22, 185-195.

Chen, X., Zhang, C., Zou, R., Zhou, K., Stephanopoulos, G., and Too, H. P. (2013). Statistical Experimental Design Guided Optimization of a One-Pot Biphasic Multienzyme Total Synthesis of Amorpha-4,11-diene. PLoS ONE 8, e79650.

Chen, Y., Kim, J. K., Hirning, A. J., Josić, K., and Bennett, M. R. (2015). Emergent genetic oscillations in a synthetic microbial consortium. Science 349, 986.

Espah Borujeni, A., Channarasappa, A. S., and Salis, H. M. (2014). Translation rate is controlled by coupled trade-offs between site accessibility, selective RNA unfolding and sliding at upstream standby sites. Nucleic Acids Res 42, 2646-2659.

Goering, A. W., Li, J., McClure, R. A., Thomson, R. J., Jewett, M. C., and Kelleher, N. L. (2016). In Vitro Reconstruction of Nonribosomal Peptide Biosynthesis Directly from DNA Using Cell-Free Protein Synthesis. ACS Synthetic Biology.

Goers, L., Freemont, P., and Polizzi, K. M. (2014). Co-culture systems and technologies: taking synthetic biology to the next level. Journal of the Royal Society, Interface/the Royal Society 11.

Groβkopf, T., and Soyer, O. S. (2014). Synthetic microbial communities. Current Opinion in Microbiology 18, 72-77.

Hall, B. G., Acar, H., Nandipati, A., and Barlow, M. (2014). Growth rates made easy. Molecular biology and evolution 31, 232-238.

Kazuta, Y., Matsuura, T., Ichihashi, N., and Yomo, T. (2014). Synthesis of milligram quantities of proteins using a reconstituted in vitro protein synthesis system. Journal of Bioscience and Bioengineering 118, 554-557.

Li, J., Gu, L., Aach, J., and Church, G. M. (2014). Improved Cell-Free RNA and Protein Synthesis System. PLoS ONE 9, e106232.

Lopez-Gallego, F., and Schmidt-Dannert, C. (2010). Multi-enzymatic synthesis. Curr Opin Chem Biol 14, 174-183.

Lu, F., Smith, P. R., Mehta, K., and Swartz, J. R. (2015). Development of a synthetic pathway to convert glucose to hydrogen using cell free extracts. International Journal of Hydrogen Energy 40, 9113-9124.

Manen, D., and Caro, L. (1991). The replication of plasmid pSC101. Molecular Microbiology 5, 233-237.

Matsubayashi, H., and Ueda, T. (2014). Purified cell-free systems as standard parts for synthetic biology. Current Opinion in Chemical Biology 22, 158-162.

Mutalik, V. K., Guimaraes, J. C., Cambray, G., Lam, C., Christoffersen, M. J., Mai, Q. A., Tran, A. B., Paull, M., Keasling, J. D., Arkin, A. P., et al. (2013). Precise and reliable gene expression via standard transcription and translation initiation elements. Nature methods 10, 354-360.

Niederholtmeyer, H., Sun, Z. Z., Hori, Y., Yeung, E., Verpoorte, A., Murray, R. M., and Maerkl, S. J. (2015). Rapid cell-free forward engineering of novel genetic ring oscillators. eLife 4, e09771.

Pandey, K. (2013). Macromolecular inhibitors of malarial cysteine proteases—An invited review. Journal of Biomedical Science and Engineering 6, 11.

Pardee, K., Green, Alexander A., Ferrante, T., Cameron, D. E., DaleyKeyser, A., Yin, P., and Collins, James J. (2014). Paper-Based Synthetic Gene Networks. Cell 159, 940-954.

Redzynia, I., Ljunggren, A., Bujacz, A., Abrahamson, M., Jaskolski, M., and Bujacz, G. (2009). Crystal structure of the parasite inhibitor chagasin in complex with papain allows identification of structural requirements for broad reactivity and specificity determinants for target proteases. FEBS Journal 276, 793-806.

Rosano, G. L., and Ceccarelli, E. A. (2014). Recombinant protein expression in Escherichia coli: advances and challenges. Frontiers in Microbiology 5.

Salis, H. M. (2011). The Ribosome Binding Site Calculator. In Methods in enzymology, V. Christopher, ed. (Academic Press), pp. 19-42.

Schultheisz, H. L., Szymczyna, B. R., Scott, L. G., and Williamson, J. R. (2008). Pathway Engineered Enzymatic de Novo Purine Nucleotide Synthesis. ACS Chemical Biology 3, 499-511

Scott, S. R., and Hasty, J. (2016). Quorum Sensing Communication Modules for Microbial Consortia. ACS Synth Biol.

Shimizu, Y., and Ueda, T. (2010). PURE Technology. In Cell-Free Protein Production: Methods and Protocols, Y. Endo, K. Takai, and T. Ueda, eds. (Totowa, NJ: Humana Press), pp. 11-21.

Shong, J., Jimenez Diaz, M. R., and Collins, C. H. (2012). Towards synthetic microbial consortia for bioprocessing. Curr Opin Biotechnol 23, 798-802.

Takahashi, M. K., Hayes, C. A., Chappell, J., Sun, Z. Z., Murray, R. M., Noireaux, V., and Lucks, J. B. (2015). Characterizing and prototyping genetic networks with cell-free transcription-translation reactions. Methods (San Diego, Calif.) 86, 60-72.

Teague, B. P., and Weiss, R. (2015). Synthetic communities, the sum of parts. Science 349, 924.

Tsuji, G., Fujii, S., Sunami, T., and Yomo, T. (2016). Sustainable proliferation of liposomes compatible with inner RNA replication. Proceedings of the National Academy of Sciences 113, 590-595.

Wang, H. H., Huang, P.-Y., Xu, G., Haas, W., Marblestone, A., Li, J., Gygi, S. P., Forster, A. C., Jewett, M. C., and Church, G. M. (2012). Multiplexed in Vivo His-Tagging of Enzyme Pathways for in Vitro Single-Pot Multienzyme Catalysis. ACS Synthetic Biology 1, 43-52.

Wu, G., Yan, Q., Jones, J. A., Tang, Y. J., Fong, S. S., and Koffas, M.A.G. Metabolic Burden: Cornerstones in Synthetic Biology and Metabolic Engineering Applications. Trends in biotechnology 34, 652-664.

### Supplementary Information

### Investigate Strategies to Control Protein Expression Levels Using Fluorescent Protein Consortia

Design and Experimental Analysis of Fluorescent Protein Consortia

In classical preparation of multi-protein complexes, proteins are individually purified, and then combined to achieve their required concentrations. Conversely, the one-shot approach enables co-expression and co-purification of all the proteins without subsequent combining steps. Therefore, it is important to modulate the expression level of each protein in the consortium. This way, the purification yield of each factor will match the required concentration of each protein.

To start, we created bacterial consortia expressing four different fluorescent proteins (each tagged with 6x-His in the C-end). The design of these consortia accounted for variables controlling protein expression, including relative densities of each strain, and rates at which the proteins are transcribed and translated. These variables were incorporated into a mathematical model that was used to predict protein expression levels (see Section 1.2).

First, three consortia were designed to modulate protein yield through relative strain densities. In these consortia, densities of CFP and GFP strains were one order of magnitude lower (consortium A), equal (consortia B), or one order of magnitude higher (consortium C) when compared to the densities of mCherry and mOrange strains (FIG. 1B). Moreover, we assumed that the high copy number plasmid gave rise to a 10 fold increase in translation rate of mOrange in consortium B when compared to the expression level when mOrange is coded by a low copy number plasmid in consortium L¹. Similarly, we considered that a modified RBS for GFP gave rise to 10 fold decrease in translation rate in consortium W when compared to the original RBS in consortium B. The predicted RBS strengths for the two RBSs were 4242.25 and 502.52 a.u. based on The RBS Calculator².

According to the model, protein levels in the consortia can be controlled by changing the relative density of each strain in the consortia (FIG. 5A and FIG. 1B) and by modifying transcription or translation rates of specific proteins (FIG. 1C). We confirmed the modeling results by testing these parameters. First, we experimentally established the consortia A, B, and C using four BL21(DE3)-pLysS strains transformed with each fluorescent protein cloned in a high copy number plasmid with a C-end 6x-His-tag for Immobilized Metal Affinity Chromatography (IMAC) purification (FIG. 6A). Each strain was grown overnight and used to establish consortia A, B, and C by mixing the strain at the indicated ratios (FIG. 1B). Consistent with predicted results, the total expression levels of each protein changed proportionally to the initial relative density of each strain in the consortium. Through these experiments, we established the control of protein expression using relative strain densities in bacterial consortia.

Next, we experimentally established consortium L by cloning mOrange in a low copy number plasmid, and consortium W by modifying the RBS sequence controlling GFP expression (FIGS. 6B and 6C). For these consortia, we used the same initial relative densities of consortium B. In agreement with model results, only GFP fluorescence levels in consortium W and mOrange fluorescence levels in consortium L decreased, proportionally to the relative RBS strength and plasmid copy number (FIG. 1C).

In addition, we investigated if purification procedures can disrupt the ratio between expression levels of each protein. To this end, we analyzed yields of each fluorescent protein from consortia A, B, and C following purification with the one-shot procedure (FIG. 1D, top). We observed that the amounts of purified proteins matched the relative densities of the strains in each consortia. The highest levels of CFP and GFP were generated by consortium A, where the strains coding for these genes were present at high densities. Moreover, the yield of each protein correlated with both the expression levels in consortia (FIG. 1B) and the predicted results by the model (FIG. 1C). In addition, we established consortia Aw, Bw and Cw, where the relative strain densities were the same as consortia A, B and C, but the strain coding for GFP was modified using the weak RBS (FIG. 2D, bottom). After one-shot purification procedure, we observed a specific decrease of GFP yield among the consortia, without significant changes in the yield of other proteins. Together, these data confirmed that protein expression level in consortia can be controlled by adjusting relative densities of the strains and tuning of coupled transcription-translation activity for each gene. Moreover, the expression levels of proteins in the consortia correlated with the concentrations of the proteins after one-shot purification.

Mathematical Modeling of Bacterial Consortia That Express Fluorescent Proteins

We formulate a system of ordinary differential equations to model the production of fluorescent proteins by the consortia (FIG. 1). Specifically, bacterial growth is modeled using the classical Monod equation by assuming that bacteria compete for a single nutrient. This assumption is likely true because all bacterial strains are modified based on the same species. We also assume that synthesis rate constants of all fluorescent proteins are the same, except for cases when plasmid copy number or RBS are modified. This assumption holds because the fluorescent proteins are expressed using the same promoter.

$$\begin{matrix}\frac{dS}{dt} = - {k}_{c} {k}_{g} ( {x}_{1} + {x}_{2} + {x}_{3} + {x}_{1} ) \frac{S}{K + S} & ( Eq . 1 ) \\ \frac{{dx}_{i}}{dt} = {k}_{g} {x}_{i} \frac{S}{K + S} & ( Eq . 2 ) \\ \frac{dPi}{dt} = {k}_{s} - {k}_{g} \frac{S}{K + S} {P}_{i} - {k}_{d} {P}_{i} & ( Eq . 3 )\end{matrix}$$

Where k_{c} represents the consumption rate constant of nutrient (nM cell⁻¹), k_{g} represents the basal growth rates of bacteria (min⁻¹), xᵢ represents the densities of bacterial strain i (cell), S represents the nutrient (nM), Pᵢ represents the fluorescent protein (nM), kₛ represents the synthesis rate constant (nM min⁻¹), and k_{d} represents the degradation rate constant (min⁻¹). kₛ is adjusted based on the known difference between the genetic constructs. Specifically, high copy number plasmid concentration is ten times higher than low copy number plasmid¹. The initiation rates of modified RBS is eight times less than the original RBS (see Section 1.1 and FIGS. 6B and 6C). k_{g} is set at 0.02 min⁻¹. k_{d} is set at 0.001 min⁻¹ because the fluorescent proteins are relatively stable inside bacteria.
Creation of Compatible Plasmids pIURAH, pIURCM and pIURKL for Cloning of TraM Genes

For the development of TraMOS, we utilized the backbones of pET15b (Novagen), pLysS (Novagen), and a pSC101 plasmids¹ to create three plasmids with the same promoter region, cloning site, and transcription termination, but different selection markers and replication origins (FIG. 7).

First, pET15b (Ampicillin^{R}, ColE1 replication origin, constitutive lad expression) was digested with XhoI and XbaI to remove the RBS and 6x-His tag coding sequence. The His-tag was removed because a subset of TraM genes were to be tagged on the C-end, but the original configuration of pET15b only allowed N-end 6x-His tag cloning. Next, using Gibson cloning, we ligated a new cloning site restoring the RBS sequence and adding restriction sites for NsiI and PacI restriction enzymes. The resulting vector formed the first plasmid of our pIUR series, termed pIURAH (pIUR Amp^{R}, High copy number).

Next, pLysS plasmid (Chloramphenicol^{R}, p15A replication origin, expressing T7 lysozyme) was digested using SalI and XhoI, while pSCTet-T7 plasmid (Kanamycin^{R}, SC101 replication origin) was digested using BglI and AvrII. Then, the fragment containing promoter, cloning site, and terminator was amplified from pIURAH using primers pairs that contained complementary regions to the digested plasmids pLysS or pSC101. The amplified fragment was then inserted into the digested plasmids through Gibson cloning. This way, we created pIURCM (pIUR Cm^{R}, Medium copy number) from pLysS and pIURKL (pIUR Km^{R}, Low copy number) from pSC101. Each of the plasmids contained the features of the original plasmids plus the hybrid PT7/lacO, the RBS sequence upstream of the unique NsiI and PacI sites, and the T7 terminator region.

As a result, we constructed plasmids with high, medium, and low copy number (pIURAH, pIURCM and pIURKL, respectively) with compatible replication origins, so they can be simultaneously maintained inside a single cell. Each plasmid has the same regulatory region and cloning site, facilitating the insertion of the TraM genes by Gibson cloning.

### Design of TraMOS Consortia

34-Strain TraMOS

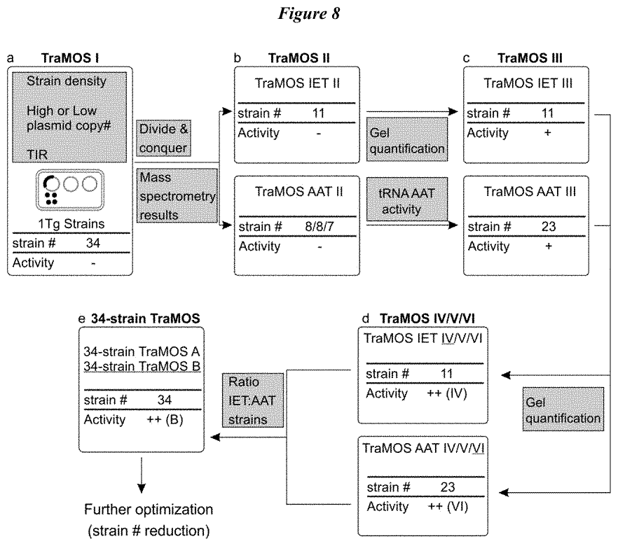

All 34 strains were generated by co-transforming BL21(DE3) using pIURAH, pIURCM and pIURKL. Each strain of this consortium coded for a single TraM gene that was cloned into either pIURAH or pIURKL (1Tg strains, Table S3). For example, strain 1Tg metG expressed the methionyl-tRNA amino acyl transferase from the pIURAH plasmid plus the non-modified (empty) pIURCM and pIURKL. Strain 1Tg aspS expressed aspartyl-tRNA amino acyl transferase from the pIURKL plasmid plus non-modified pIURAH and pIURCM. Consequently, all 34 strains carried the three plasmids. A summary of the steps taken to optimize the 34-strain consortia is described in the next sections (also shown in FIG. 8).

Creation of TraMOS I, TraMOS II and TraMOS III

TraMOS I was designed using fixed strain densities of each strain as per the plasmid was high- or low-copy number. Therefore, strain relative densities in consortium was of 0.22% for high copy number or 2.17% for low copy number. We also predicted the translation initiation rates (TIR) of each gene cloned in pIURAH, pIURKL and pIURCM using The RBS Calculator (Table S6). We used these predicted rates to correct the strain densities volumes when the predicted TIR was lower than 10000 au and coded in low copy number plasmid (Table S6). This initial approach generated TraMOS I with very low expression activity (not shown). To understand the issue, we analyzed the protein composition of TraMOS I by mass spectrometry (not shown). The results were used to correct the relative densities of the strains, using the concentrations reported in a previous work³. Based on the results, we established 4 new subconsortia: IET TraMOS II (11-strains), AAT1 TraMOS II, AAT2 TraMOS II (each with 8 different AAT-strains) and AAT3 TraMOS II (7-strains) (Table S6). Again, these preparations yielded very low in vitro translation activities.

To identify the problem and to optimize the consortia, we took several steps to understand the functionality of the translation factors. For IET factors, we purified them separately and created a Control IET that was functional. For a comparative analysis, we ran the Control IET mixture and TraMOS II fraction on SDS-PAGE and quantified the bands corresponding to each factor. This way, using the Control IET as the target, we measured the amount of each protein in TraMOS II and used the data to calculate the initial relative densities of the strains in the subsequent consortia (Table S6).

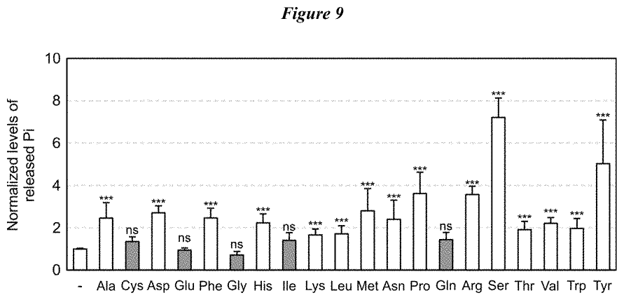

Because AAT genes have very similar molecular weights, we could not apply the above strategy to these factors. To this end, we measured the activity of each enzyme using a colorimetric method⁴. This method relies on the generation of pyrophosphate from ATP, which is a required step in the conjugation of tRNA-amino acyl catalyzed by the enzyme. Pyrophosphate is then converted to free inorganic phosphate (Pi). Therefore, the levels of Pi represent a direct measurement of AAT activity. Using tRNA and the specific amino acid, we determined activity of all the enzymes in the three subconsortia (FIG. 9). We observed that activity of Cys, Gly, Ile and Gln-AATs were very low and comparable to the control. Therefore, we aimed to increase the relative densities of these AATs.

With these new insights, we developed two subconsortia, TraMOS IET III and TraMOS AAT III (Table S6), as presented in the main text. These preparations generated moderate expression activities when compared to the Control IET and AAT (FIG. 2B). To improve both IET and AAT TraMOS activities, we designed three new IET and three new AAT subconsortia to test their activities separately.

Creation of Optimized IET Subconsortia

For the optimization of IET subconsortia, we compared the TraMOS IET III with the Control IET by SDS-PAGE. IET IV was then designed based on the quantification of bands on SDS-PAGE for each factor, which guided the readjustment of relative strain densities. In addition, we designed TraMOS IET V and VI because initiation and elongation factors (particularly EF-G, EF-Ts and EF-Tu) are required at a higher ratio relative to termination factors. Using the design of TraMOS IET IV as a starting point, we increased the relative initial densities of initiation factors-strains by 50% and decreased EF's strains by the same factor to produce TraMOS IET V. Similarly, we designed TraMOS IET VI by increasing both initiation and elongation factors' strains by 25%, while reducing termination factors' strains by 50% (Table S6). Of these three preparations, IET VI resulted in the highest activity (FIG. 2C). We also observed the dependence between activity of mixture TraMOS IET IV:TraMOS AAT III and the ratio of total protein between IET and AAT preparations (FIG. 10). The ratio (calculated as the ng of protein in IET fraction/ng protein in AAT fraction) for the mixture TraMOS IET IV:Control AAT shown in FIG. 2C was 6. Increasing the ratio TraMOS IET IV:Control AAT to 28 increased the expression activity by 6 fold (FIG. 10A). Moreover, we observed that modifying the ratio TraMOS IET IV:TraMOS AAT III to 14 and 21 increased the expression activity of the TraMOS-based preparations, comparable to the mixture with the TraMOS IET IV:Control AAT at ratio 28 (FIG. 10B). Therefore, IET factors have to be present at higher concentrations than the AAT factors in the final product.

Creation of Optimized AAT Subconsortia

The optimization of AAT subconsortia was approached differently. Based on the requirements of each AAT factor in a previous work⁵, we adjusted the relative volumes of the strains based on their activities and protein-gel quantification (the latter, whenever possible considering that some of the AAT factors cannot be separated in SDS-PAGE due to similarities in their molecular weights). The resulting subconsortium was termed TraMOS AAT IV. We also designed another subconsortium using the same method (TraMOS AAT V), but replaced the strains coding for 6 AAT factors in low copy number plasmids by strains coding for these genes in high copy number plasmids. Finally, we created another subconsortium (TraMOS AAT VI), in which we utilized the same strains as in TraMOS AAT V, but with adjusted composition. For this, the relative densities of strains in TraMOS AAT VI were calculated based on the required protein levels, plasmid copy number, and TIR. Specifically, we first estimated the relative protein concentration of each factor in the PURE system (R_{Pure}). Following this step, we calculated a factor T for each factor by multiplying the relative plasmid copy number (values of 100 for high and 10 for low) times their predicted TIR. We then normalized these factors using the maximal T (corresponding to glyS-C in high copy number plasmid). Finally, we calculated the relative strain density for the consortium correcting the density in TraMOS AAT III by the factor estimated with the formula R_{Pure}/T. With this information, we experimentally established the consortia (Table S6). According to our results, TraMOS AAT VI resulted in the highest activity by a factor of approximately 1.5 times relative to the control (FIG. 1D).

Establishment of Functional 34-Strain Consortia

Finally, we established 34-strain consortia A and B by preparing IET IV and AAT VI subconsortia with the optimized relative densities and strains, and then combined them IET IV:AAT VI with ratios 30:1 (34-strain TraMOS A) or 60:1 (34-strain TraMOS B). The resulting consortia were inoculated into LB media, followed by induction and one-shot purification of TraMOS (Table S7).

18-Strain TraMOS

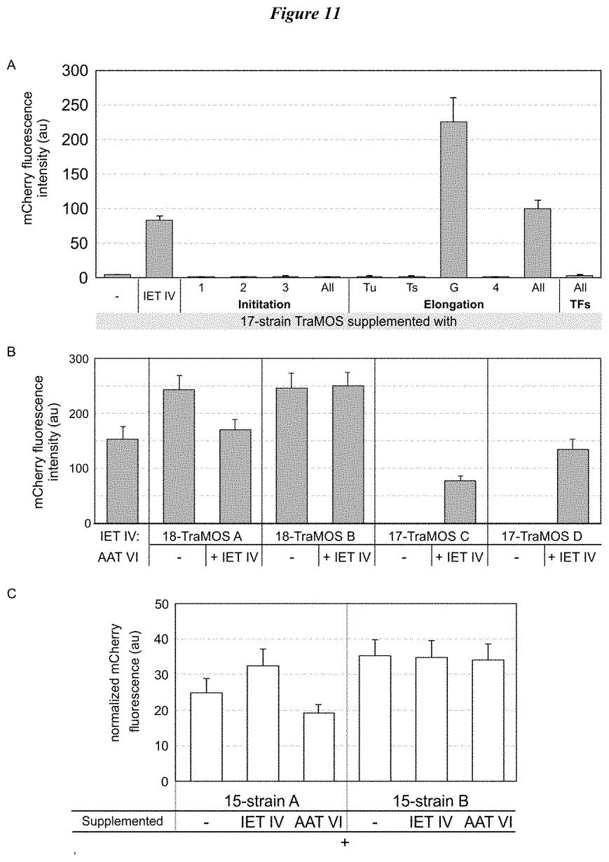

We first created strains that simultaneously expressed two TraM genes. To do this, we co-transformed BL21(DE3) strain using both pIURAH and pIURKL plasmids that expressed TraM genes, together with the empty plasmid pIURCM (2Tg strains). The composition of the 18-strain consortia is shown in the Table S8. We utilized the design of the 34-strain consortium to guide the design of the 2Tg strains. Specifically, the TraM genes expressed in strains at the highest densities in the 34-strain consortium were combined into a single 2Tg strain. For example, in 34-strain consortium, the two strains required at higher densities are 1Tg EF-Tu (high copy number) and 1Tg EF-Ts (low copy number). Therefore, one strain 2Tg IET 2 was created carrying both EF-Tu and EF-Ts genes in high and low copy number plasmids respectively. Following this logic, we created the remaining 16 2Tg strains (Table S8). We also considered grouping the genes functionally whenever possible. Therefore, we combined all the IET factors in five 2Tg IET strains and 22 AAT factors in eleven 2Tg AAT strains. One strain (2Tg IET 4) coded for both EF-4 (in low copy number plasmid) and alaS AAT gene (in high copy number plasmid). Using these strains, we established a 17-strain consortium that resulted in a non-functional mixture. The activity was restored, however, following supplementation with purified EF-G (FIG. 11A). Based on this result, we created four consortia: two of them supplemented the 17-strain consortium with the 1Tg EF-G strain at two different densities (18-strain consortia A and B, Table S8). Additionally, we created 17-strain consortia C and D, in which we increased relative densities of 2Tg IET 6 strain (expressing EF-G and IF3). After preparation of TraMOS, we determined that preparation from 2Tg TraMOS B consortium (supplemented with 8% relative density of the 1Tg EF-G coding strain) was functional, resulting in the functional 18-strain consortium used hereafter (FIG. 11B).

15-Strain TraMOS

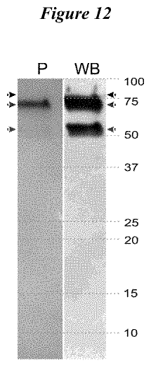

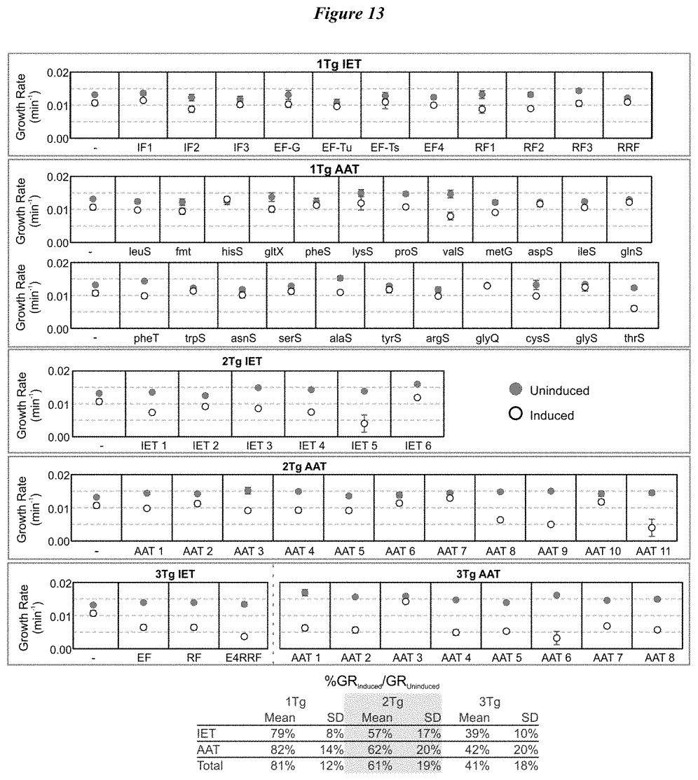

To further decrease the number of strains in the consortia, we created strains coding for three TraM genes simultaneously (3Tg strains) by co-transforming BL21(DE3) bacteria with pIURAH, pIURCM, and pIURKL plasmids, each expressing one TraM gene. We designed the 3Tg strains based on the design of the 18-strain consortia and grouped initiation, elongation, termination or AAT factors together whenever possible (Table S9). This way, we designed strains that expressed the three initiation factors (3Tg IET), elongation factors Tu-Ts-G (3Tg EF), and release factors (3Tg RF). We also created a fourth strain coding for EF-4 (required at lower concentration compared to the other elongation factors), RRF, and EF-G (3Tg E4RRF). In addition, we designed eight 3Tg AAT strains, each coding for three distinct AAT genes, except for 3Tg AAT 6, which coded for alaS in both pIURAH and pIURCM (since alaS is the AAT required at higher levels). We were not able to obtain colonies for the 3Tg IET strains. In addition, strain 3Tg AAT 8 that expressed cysS, glyS, and thrS presented a very low growth rate upon induction and low expression level of glyS (FIGS. 12 and 13). Because of this, we supplemented the 3Tg strains with three 2Tg strains: two of them carrying the IFs (2Tg IET3 and 2Tg IET 6) and one coding for glyS (2Tg AAT 4), plus one 1Tg strain coding for EF-G. Relative densities of the IET and AAT strains were calculated based on the 18-strain consortium (Tables S8 and S9). We established two consortia with different ratios for IET to AAT coding strains, termed 15-strain TraMOS A and 15-strain TraMOS B. After determining expression activities of the resulting protein mix, we defined the latter as the 15-strain TraMOS in the main text (FIG. 11C). We also modified the protocol for induction of 15-strain TraMOS. We observed a marked reduction of the growth rate in ten of the 3Tg strains after induction with IPTG, where average growth rate of the induced 3Tg strains was 41%±18% of the original (uninduced) growth rate (FIG. 13). We also observed a general decrease in the growth rates of most of the 2Tg strains, although the impact was less significant (overall induced growth rate was 61%±19% of the uninduced growth rate). The growth rates of the 1Tg strains were also affected upon induction, but at a lower extent (81%±12% of uninduced growth rate). Because of these observations, we increased the number of cells in the inoculum and extended the time of induction when producing 15-strain TraMOS to maximize protein yield (see Methods).

Chagasin Library Design and Development

Cys-protease inhibitors from parasites such as Trypanozoma cruzi or Plasmodium falciparum are implicated in pathogenesis⁶. Interaction of the inhibitors with the protease is mediated through a number of amino acids in three loops in the inhibitor (termed BC, DE and FG) with amino acids surrounding the protease's active site⁷ (FIG. 14A). A majority of the amino acids in these loops are highly conserved in this inhibitor family, although a number of them present some variability (FIG. 14B). We hypothesized that the variable positions involved in protein-protein interaction can affect the activity of the inhibitor. Consequently, we designed a set of degenerated primers (Table S1) to introduce variation in loops DE (positions 64, 65 and 67) and FG (positions 91, 92, 93 and 99, FIG. 14B). In addition, two primers were designed to introduce two variants (Thr or Gly) in position 31 of loop BC. We designed the primers in order to introduce degenerated codons by maximizing the introduction of amino acids present in at least one sequence of the inhibitor family, but minimizing introduction of amino acids that are not present in any of the natural sequences (Table S1). This way, our strategy introduced a number of mutations at the selected positions (FIG. 14C), creating more than 160,000 possible variants.

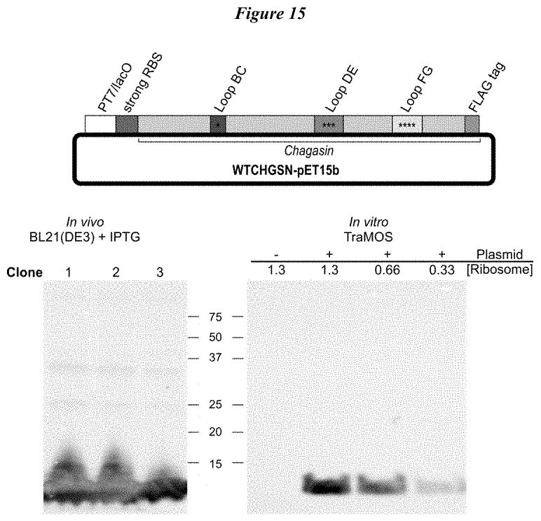

The WT chagasin DNA sequence (derived from the amino acid sequence Q966X9.1) was synthesized by incorporating a strong RBS sequence (designed to maximize translation rate), an octapeptide FLAG-tag sequence in the C-end, and a synthetic terminator, T7U-T7 TΦ⁸. The synthesized fragment was inserted into pET15b plasmid (digested Xba I/EcoRI) using Gibson Assembly, generating the plasmid WTCHGSN-pET15b (GenBank accession#KX765180). We produced chagasin both in vivo and in vitro, as demonstrated by western blot using anti-FLAG antibody (FIG. 15).

Using WTCGSHN-pET15b as the template, we generated four PCR fragments using the degenerated primers, covering overlapping regions of the full length chagasin gene. Two fragments covered the BC loop, each with one of the two possible variants (Thr31 or Gly31), one fragment introduced mutations in loop DE and the fourth carried mutations in loop FG. All these fragments, together with the XbaI/HindIII-digested WTCHGSN-pET15b plasmid, were combined in a single Gibson Assembly reaction to randomly generate chagasin variants. The resulting library was transformed into E. coli, obtaining approximately 10⁴ clones after a single transformation event. We randomly sequenced 24 clones and observed that the sequences were highly variable with the expected mutations at the target positions (FIG. 14D). We then selected random clones from this library to screen for their inhibitory capacity over Cys-protease activity (FIG. 14E).

Mathematical Model for 34- and 18-Strain TraMOS

Predicting quantitative outputs from design inputs is an important feature of engineered systems. For an engineered consortium, a model that uses design inputs such as plasmid copy number would be a valuable tool for the a priori design of a system that yields specific protein concentrations. To this end, we create a set of equations that models the inter- and intra-cellular interaction in order to lay a foundation for predicting the protein yields of engineered, multi-strain consortia.

To begin, we compared quantified TraM protein levels in three biological replicates of 34- and 18-strain consortia from mass spectrometry (FIG. 3C). In all cases, we observe Pearson correlations, r, of greater than 0.95 (FIG. 17A). This high correlation indicates that stochastic processes have little effect on observed protein outputs, which may be attributable to the design principles followed during the creation of the constituent strains⁹. Due to this low stochastic variation, a deterministic model can be used to describe the system.

To predict protein yields from knowledge of the way the consortium is engineered, the model includes processes at both the population and molecular levels. To begin, the model predicts how individual strains grow while competing for resources with other strains in the consortia (Eqn. 4). The number of cells, N, for the ith strain in the consortium grows exponentially at rate, r. However, further growth is inhibited as the total number of cells in the consortium reach the cultures carrying capacity, K.

On the molecular level, each cell carries multiple copies of the gene expressed by each strain, Dᵢ, (Eqn 5). The number of genes present in the consortium is determined by the plasmid copy number engineered into each strain, Cᵢ and is directly proportional to the number of cells for each strain. Finally, the protein output of strain, Pᵢ, is determined by a synthesis rate, αᵢ, and degradation rate, Δᵢ, which incorporates multiple cellular processes such as transcription and translation (Eqn 6). The synthesis of protein is dependent on the amount of genes present and the length of the gene. Degradation is solely dependent on the amount of protein.

$$\begin{matrix}\frac{{dN}_{i}}{dt} = {r}_{i} {N}_{i} ( 1 - \frac{{\sum }_{i = 1}^{n} {N}_{i}}{K} ) & ( Eq . 4 ) \\ \frac{{dD}_{i}}{dt} = {C}_{i} \frac{{dN}_{i}}{dt} & ( Eq . 5 ) \\ \frac{{dP}_{i}}{dt} = \frac{{α}_{i}}{{l}_{i}} {D}_{i} - {Δ}_{i} {P}_{i} & ( Eq . 6 )\end{matrix}$$

The growth rates r for the strains following IPTG induction are calculated based on experimental results (FIG. 13). Furthermore, we define the plasmid copy number, Cᵢ, as 10 times larger for high copy number strains and 2.5 times larger for medium copy number strains when compared to low copy number strains. These numbers arise from previous measurements of plasmids per cell for each origin of replication^{1, 10}.

Measuring the in vivo synthesis and degradation for each protein is not feasible for the TraMOS system. Instead, we train the model in silico using the average mass spectrometry data for the 34-strain consortium. Using MATLAB's stiff ODE solver, we first set αᵢ and Δᵢ to one and use the relative initial cell density (as a percentage of the initial inoculum with OD₆₀₀ of 0.01) as the initial condition for each strain, Nᵢ(0). We then iterate the model for each strain to simulate the growth and protein production of the consortium over time.

Using the protein concentration achieved at steady state, we then create a prediction for the protein output of the 34-strain consortium not taking into account differences in synthesis and degradation. Comparing these values to actual mass spectrometry values, we quantitatively determine the synthesis rate that would achieve perfect correlation (r=1) between predictive and measured protein outputs while leaving degradation rates equal to 1 (FIG. 17B, left). In this way, we calculated an estimate of this cellular phenomenon.

To further test the validity of this approach, we extend the model to the 18-strain consortia, using synthesis rates previously calculated. Here, the growth rate, rᵢ, is recalculated for each 2Tg strain. The 18-strain model uses the previously described equation for modeling population dynamics of the strain (Eqn 4). Similarly, the number of genes for each protein and the total protein yield uses the same equations for the 2Tg as for the 1Tg strain. However, now each 2Tg strain is modeled with two gene copy equations, D₁ᵢ, and D₂ᵢ (Eqn 7 and 8) that are both directly proportional to cell number of the strain. Furthermore, there are two protein yield equations, P₁ᵢ and P₂ᵢ, which uses the calculated synthesis rates and the DNA copies of their respective genes (Eqn 9 and 10).

$$\begin{matrix}\frac{{dD}_{1 i}}{dt} = {C}_{1 i} \frac{{dN}_{i}}{dt} & ( Eq . 7 ) \\ \frac{{dD}_{2 i}}{dt} = {C}_{2 i} \frac{{dN}_{i}}{dt} & ( Eq . 8 ) \\ \frac{{dP}_{1 i}}{dt} = \frac{{α}_{i 1}}{{l}_{1}} {D}_{1 i} - Δ {P}_{1 i} & ( Eq . 9 ) \\ \frac{{dP}_{2 i}}{dt} = \frac{{α}_{i 2}}{{l}_{2}} {D}_{2 i} - Δ {P}_{2 i} & ( Eq . 10 )\end{matrix}$$

Using the same in silico method as described above, the predicted protein output at steady state is compared to measured values of the 18-strain consortium (FIG. 17B, right). The predictive model shows high predictive capabilities as it correlates well with measured values (r=0.65).

This model lays a foundation for predicting protein yields from engineered, multi-strain consortia. For TraMOS, where the proportions of proteins relative to one another are key to the activity of the whole, this model is a valuable tool in future optimization and modification of the consortia .

### SUPPLEMENTARY REFERENCES

1. Manen, D. & Caro, L. The replication of plasmid pSC101. Molecular Microbiology 5, 233-237 (1991).
2. Salis, H. M. in Methods in Enzymology, Vol. Volume 498. (ed. V. Christopher) 19-42 (Academic Press, 2011).
3. Shimizu, Y., Kanamori, T. & Ueda, T. Protein synthesis by pure translation systems. Methods (San Diego, Calif.) 36, 299-304 (2005).
4. Cestari, I. & Stuart, K. A spectrophotometric assay for quantitative measurement of aminoacyl-tRNA synthetase activity. Journal of biomolecular screening 18, 490-497 (2013).
5. Kazuta, Y., Matsuura, T., Ichihashi, N. & Yomo, T. Synthesis of milligram quantities of proteins using a reconstituted in vitro protein synthesis system. Journal of Bioscience and Bioengineering 118, 554-557 (2014).
6. Pandey, K. Macromolecular inhibitors of malarial cysteine proteases—An invited review. Journal of Biomedical Science and Engineering 6, 11 (2013).
7. Hansen, G. et al. Structural basis for the regulation of cysteine-protease activity by a new class of protease inhibitors in Plasmodium. Structure (London, England: 1993) 19, 919-929 (2011).
8. Mairhofer, J., Wittwer, A., Cserjan-Puschmann, M. & Striedner, G. Preventing T7 RNA Polymerase Read-through Transcription—A Synthetic Termination Signal Capable of Improving Bioprocess Stability. ACS Synthetic Biology 4, 265-273 (2015).
9. Elowitz, M. B., Levine, A. J., Siggia, E. D. & Swain, P. S. Stochastic Gene Expression in a Single Cell. Science 297, 1183-1186 (2002).
10. Chang, A. C. & Cohen, S. N. Construction and characterization of amplifiable multicopy DNA cloning vehicles derived from the P15A cryptic miniplasmid. J Bacteriol 134, 1141-1156 (1978).
11. Hanson, R. Jmol—a paradigm shift in crystallographic visualization. Journal of Applied Crystallography 43, 1250-1260 (2010).

Table S1. List of oligonucleotides used in this study. For the primers used in chagasin mutagenesis (Information Section 4 and FIG. 14C), sequences that introduce degenerate codons are shown in capital letters.

TABLE S1

| | | Target | |
|---|---|---|---|
| Goal | Gene | plasmid | Sequence (5′-3′) |
| Fluorescent | GFPstrong-6xHis | pET15b | aaataattttgtttaactttaagaaggagatataccatgaagttaaa |
| protein | tag | | gatctgcgtaaacctttcgggctttgttagcagccggatcctcgatt |
| consortia | | | agtgatgatgatgatgatggtatagttcatccatg |
| | CFP-6xHis tag | pET15b | aaataattttgtttaactttaagaaggagatataccatgcgtaaagg |
| | | | agaagaacttttctcctttcgggctttgttagcagccggatcctcga |
| | | | ttagtgatgatgatgatgatgtttgtatagttcatc |
| | mOrange high- | pET15b | aaataattttgtttaactttaagaaggagatataccatggttagtaa |
| | 6xHis tag | | aggagaagaaaaccctttcgggctttgttagcagccggatcctcgat |
| | | | tagtgatgatgatgatgatgtgcggtaccagaacct |
| | mOrange | pIURKL | ctttaagaaggagatataccatgcaatggttagtaaaggagaagaaa |
| | low-6xHis tag | | aca |
| | mCherry-6xHis | pET15b | aaataattttgtttaactttaagaaggagatataccatggtgagcaa |
| | tag | | gggcgaggaggatcctttcgggctttgttagcagccggatcctcgat |
| | | | tagtgatgatgatgatgatgcttgtacagctcgtcc |
| Insertion | RBS_GFPweak | C.GFP- | cggataacaattcccctctagactacaacttcttctcaatcagaagg |
| of weak RBS | | pET15b | agtaagtcaatgcaccataaccgaaagtagtgacaagtgttggccat |
| controlling | | | ggaacaggtagttttccagtagtgc |
| GFP | | | |
| translation | | | |
| rate | | | |
| Conversion | Stitch MCS | pET15b | gtatatctccttcttaaagttaaacaaaattatttctagaggggaat |
| pET15b to | | | tgttatccgctcagtttaactttaagaaggagatataccatgcatca |
| pIURAH | | | tcatcaccatcatttaattaaagcaggctttgttagcagccggatcc |
| | | | tcgagcatatggccgctgctttaattaaatgatggtgat |
| Creation of | pIURCM | pIURAH | tgcaggagtcgcataagggagagcgtcgacccctggatgctgtaggc |
| pIURKL | | | ataggcttggttatccatgccaacccgttccatgtgctcgccggatc |
| and | pIRUKL | | ccgcgaaattaatacgactcactata |
| pIURCM | | | |
| TraM | alaS-N | pIUR | tataccatgcatcatcatcaccatcatttaagcaagagcaccgctga |
| genes | | plasmids | gatcctcaggcgagcatatggccgctgctttaatattgcaatttcgc |
| cloning | | | gctgacccagcctttcac |
| | argS-N | pIUR | tataccatgcatcatcatcaccatcatttaaatattcaggctcttct |
| | | plasmids | ctcagaaaaagtcagcatatggccgctgctttaatacatacgctcta |
| | | | cagtctcaatacccagcgt |
| | asnS-N | pIUR | tataccatgcatcatcatcaccatcatttaagcgttgtgcctgtagc |
| | | plasmids | cgacgtactccagtggtgttggagttttcagcacggaaagtcgggcc |
| | | | gaaggtataaattttgtttccgtgctgaaaactccaacaccagccgt |
| | | | cacctggcggaattctggaagcatatggccgctgctttaatagaagc |
| | | | tggcgttacgcggagtacgtgggaa |
| | aspS-C | pIUR | ttaactttaagaaggagatataccatgcatcgtacagaatattgtgg |
| | | plasmids | acagctccgtttgaccggcgacgaaattaatgatggtgatgatgatg |
| | | | gttattctcagccttcttcacaacct |
| | cysS-C | pIUR | ttaactttaagaaggagatataccatgcatctaaaaatcttcaatac |
| | | plasmids | tctgacacgccaaaccggcgacgaaattaatgatggtgatgatgatg |
| | | | cttacgacgccaggtggtcccttgcg |
| | fmt-C | pIUR | ttaactttaagaaggagatataccatgcattcagaatcactacgtat |
| | | plasmids | tatttttgcgggtaccggcgacgaaattaatgatggtgatgatgatg |
| | | | gaccagacggttgcccggaacaaacc |
| | frr-C | pIUR | ttaactttaagaaggagatataccatgcatattagcgatatcagaaa |
| | | plasmids | agatgctgaagtaaccggcgacgaaattaatgatggtgatgatgatg |
| | | | gaactgcatcagttctgcttctttgt |
| | fusA-C | pIUR | ttaactttaagaaggagatataccatgcatgctcgtacaacacccat |
| | | plasmids | cgcacgctaccgtaccggcgacgaaattaatgatggtgatgatgatg |
| | | | tttaccacgggcttcaattacggcct |
| | glnS-N | pIUR | tataccatgcatcatcatcaccatcatttaagtgaggcagaagcccg |
| | | plasmids | cccgactaactttagcatatggccgctgctttaatactcgcctactt |
| | | | tcgcccaggtatcacgcag |
| | gltX-C | pIUR | ttaactttaagaaggagatataccatgcataaaatcaaaactcgctt |
| | | plasmids | cgcgccaagcccaaccggcgacgaaattaatgatggtgatgatgatg |
| | | | ctgctgattttcgcgttcagcaataa |
| | glyQ-C | pIUR | ttaactttaagaaggagatataccatgcatcaaaagtttgataccag |
| | | plasmids | gaccttccagggcaccggcgacgaaattaatgatggtgatgatgatg |
| | | | cttatctttgttgcacatcgggaagc |
| | glyS-C | pIUR | ttaactttaagaaggagatataccatgcattctgagaaaacttttct |
| | | plasmids | ggtggaaatcggcaccggcgacgaaattatgatggtgatgatgatgt |
| | | | tgcaacagcgaaatatccgcaacgc |
| | hisS-C | pIUR | ttaactttaagaaggagatataccatgcatgcaaaaaacattcaagc |
| | | plasmids | cattcgcggcatgaccggcgacgaaattaatgatggtgatgatgatg |
| | | | acccagtaacgtgcgcaaatgcgcgg |
| | ileS-N | pIUR | tataccatgcatcatcatcaccatcatttaagtgactataaatcaac |
| | | plasmids | cctgaatttgccgagcatatggccgctgctttaataggcaaacttac |
| | | | gtttttcaccgtcaccggc |
| | infA-N | pIUR | tataccatgcatcatcatcaccatcatttagccaaagaagacaatat |
| | | plasmids | tgaaatgcaaggtagcatatggccgctgctttaatagcgactacgga |
| | | | agacaatgcggcctttgct |
| | infB-N | pIUR | tataccatgcatcatcatcaccatcatttaacagatgtaacgattaa |
| | | plasmids | aacgctggccgcaagcatatggccgctgctttaataagcaatggtac |
| | | | gttggatctcgatgatttc |
| | infC-N | pIUR | tataccatgcatcatcatcaccatcatttaaaaggcggaaaacgagt |
| | | plasmids | tcaaacggcgcgcagcatatggccgctgctttaatactgtttcttct |
| | | | taggagcgagcaccatgat |
| | lepA-C | pIUR | ttaactttaagaaggagatataccatgcataagaatatacgtaactt |
| | | plasmids | ttcgatcatagctaccggcgacgaaattaatgatggtgatgatgatg |
| | | | tttgttgtctttgccgacgtgcagaa |
| | leuS-C | pIUR | ttaactttaagaaggagatataccatgcatcaagagcaataccgccc |
| | | plasmids | ggaagagatagaaaccggcgacgaaattaatgatggtgatgatgatg |
| | | | gccaacgaccagattgaggagtttac |
| | lysS-C | pIUR | ttaactttaagaaggagatataccatgcattctgaacaacacgcaca |
| | | plasmids | gggcgctgacgcgaccggcgacgaaattaatgatggtgatgatgatg |
| | | | ttttaccggacgcatcgccgggaaca |
| | metG-C | pIUR | ttaactttaagaaggagatataccatgcatactcaagtcgcgaagaa |
| | | plasmids | aattctggtgacgaccggcgacgaaattaatgatggtgatgatgatg |
| | | | tttcacctgatgacccggtttagcac |
| | pheS-N | pIUR | tataccatgcatcatcatcaccatcatttatcacatctcgcagaact |
| | | plasmids | ggttgccagtgcgagcatatggccgctgctttaatatttaaactgtt |
| | | | tgaggaaacgcagatcgtt |
| | pheT-C | pIUR | ttaactttaagaaggagatataccatgcataaattcagtgaactgtg |
| | | plasmids | gttacgcgaatggaccggcgacgaaattaatgatggtgatgatgatg |
| | | | atccctcaatgatgcctggaatcgct |
| | prfA-N | pIUR | tataccatgcatcatcatcaccatcatttaaagccttctatcgttgc |
| | | plasmids | caaactggaagccagcatatggccgctgctttaatattcctgctcgg |
| | | | acaacgccgccagttggtc |
| | prfB-N | pIUR | tataccatgcatcatcatcaccatcatttatttgaaattaatccggt |
| | | plasmids | aaataatcgcattagcatatggccgctgctttaatataaccctgctt |
| | | | tcaaacttgcttcgataaa |
| | prfC-N | pIUR | tataccatgcatcatcatcaccatcatttaacgttgtctccttattt |
| | | plasmids | gcaagaggtggcgagcatatggccgctgctttaataatgctcgcggg |
| | | | tctggtggaactgaacgtc |
| | proS-C | pIUR | ttaactttaagaaggagatataccatgcatcgtactagccaatacct |
| | | plasmids | gctctccactctcaccggcgacgaaattaatgatggtgatgatgatg |
| | | | gcctttaatctgtttcaccagatatt |
| | serS-C | pIUR | ttaactttaagaaggagatataccatgcatctcgatcccaatctgct |
| | | plasmids | gcgtaatgagccaaccggcgacgaaattaatgatggtgatgatgatg |
| | | | gccaatatattccagtccgttcatat |
| | thrS-N | pIUR | tataccatgcatcatcatcaccatcatttacctgttataactcttcc |
| | | plasmids | tgatggcagccaaagcatatggccgctgctttaatattcctccaatt |
| | | | gtttaagactgcggctgcg |
| | trpS-C | pIUR | ttaactttaagaaggagatataccatgcatactaagcccatcgtttt |
| | | plasmids | tagtggcgcacagaccggcgacgaaattaatgatggtgatgatgatg |
| | | | cggcttcgccacaaaaccaatcgctt |
| | tsf-C | pIUR | ttaactttaagaaggagatataccatgcatgctgaaattaccgcatc |
| | | plasmids | cctggtaaaagagaccggcgacgaaattaatgatggtgatgatgatg |
| | | | agactgcttggacatcgcagcaactt |
| | tufA-C | pIUR | ttaactttaagaaggagatataccatgcattctaaagaaaatttgaa |
| | | plasmids | cgtacaaaaccgaccggcgacgaaattatgatggtgatgatgatggc |
| | | | ccagaactttagcaacaacgcccg |
| | tyrS-C | pIUR | ttaactttaagaaggagatataccatgcatgcaagcagtaacttgat |
| | | plasmids | taaacaattgcaaaccggcgacgaaattaatgatggtgatgatgatg |
| | | | tttccagcaaatcagacagtaattct |
| | valS-C | pIUR | ttaactttaagaagagatataccatgcatgaaaagacatataaccca |
| | | plasmids | caagatatcgaaaccggcgacgaaattaatgatggtgatgatgatgc |
| | | | agcgcggcgataacagcctgctgtt |
| | Trameend_Cloner | pIUR | gatcctcgagcatatggccgctgctttaatgatggtgatgatgatg |
| | | plasmids | |
| Chagasin | Full length | pET15b | attgtgagcggataacaattcccctctagacccaggtctatacgtag |
| library | Chagasin Fw | | taaggaggtaagg |
| creation | Full length | pET15b | cgggttgctcggcagctgaatttccaccagttcgcccaccgccacgg |
| | Chagasin Rv | | tcagggtcgcgcc |
| | Mutation T | CHGSN | ctggtggaaattcagctgccgagcaacccgaccaccggctttgcgtg |
| | loop BC | WT- | gtattttgaaggc |
| | | pET15b | |
| | Mutation G | CHGSN | ctggtggaaattcagctgccgagcaacccgggcaccggctttgcgtg |
| | loop BC | WT- | gtattttgaaggc |
| | | pET15b | |
| | Reverse | CHGSN | tttgctatccggcggaaaatatttgttttccacggtaaacatgcttt |
| | | WT- | cgttcgggctttc |
| | loop BC | pET15b | |
| | Mutations | CHGSN | caaatattttccgccggatagcaaaVKGBKKggcKBSggcggcaccg |
| | loop DE Fw | WT- | aacattttcatgt |
| | | pET15b | |
| | Mutations | CHGSN | catataggtcaggttcaccgcatgggtgcccgccgctttcacggt |
| | loop DE Rv | WT- | |
| | | pET15b | |
| | Mutations | CHGSN | gggcacccatgcggtgaacctgacctatatgCRRSHTKKaccggccc |
| | loop FG Fw | WT- | gagccatVMTag |
| | | pET15b | |
| | Mutations | CHGSN | aacgactacaaagacgatgacgacaagtaaaagcttctttcagcaaa |
| | loop FG Rv | WT- | aaaccccgcgaga |
| | | pET15b | |

Table S2. Features of TraM genes. TraM genes are divided in two main functional categories, IETs (Initiation, Elongation and Termination factors), and AATs (tRNA-amino acyl transferases). Location of the 6x-His-tag is shown for each TraM gene (-N, N-end; -C, C-end). EcoGene database accession numbers are shown. Translation initiation rates (TIR) are calculated using The RBS calculator. Purity of each factor is quantified from protein gels stained with Coomassie brilliant blue.

TABLE S2

| | | | | | | calculated | |
|---|---|---|---|---|---|---|---|
| | | | Accession | length | MW | TIR^{a} | |
| | Name | Description | number | (bp) | (kDa) | (au) | Purity |
| IET | IF1-N | translational initiation factor 1 | EG10504 | 273 | 9.07 | 37461.41 | 87.6% |
| | IF2-N | translational initiation factor 2 | EG10505 | 2727 | 98.19 | 13981.19 | 87.1% |
| | IF3-N | translational initiation factor 3 | EG10506 | 597 | 21.37 | 15229.3 | 93.9% |
| | EF-G-C | translational elongation factor G | EG10360 | 2173 | 78.42 | 16440.19 | 93.1% |
| | EF-Tu-C | translational elongation factor Tu | EG11036 | 1243 | 44.08 | 28086.32 | 70.1% |
| | EF-Ts-C | translational elongation factor Ts | EG11033 | 910 | 31.25 | 1258.56 | 82.4% |
| | EF4-C | translational elongation factor 4 | EG10529 | 1858 | 67.4 | 75594.36 | 94.0% |
| | RF1-N | translational release factor 1 | EG10761 | 1137 | 41.35 | 15858.71 | 59.2% |
| | RF2-N | translational release factor 2 | EG10762 | 1152 | 42.08 | 42876.54 | 60.1% |
| | RF3-N | translational release factor 3 | EG12114 | 1648 | 60.41 | 13365.92 | n.a. |
| | RRF-C | ribosome recycling factor | EG10335 | 616 | 21.43 | 33626.04 | 90.7% |
| AAT | valS-C | Val-tRNA synthetase | EG11067 | 2914 | 109.03 | 24539.13 | 67.3% |
| | metG-C | Met-tRNA synthetase | EG10568 | 2092 | 77.09 | 5078.99 | 70.3% |
| | ileS-N | Ile-tRNA synthetase | EG10492 | 2871 | 105.13 | 46914.83 | 82.7% |
| | thrS-N | Thr-tRNA synthetase | EG11001 | 1983 | 74.85 | 56168.27 | 73.3% |
| | lysS-C | Lys-tRNA synthetase | EG10552 | 1576 | 58.43 | 24539.13 | 91.3% |
| | gltX-C | Glu-tRNA synthetase | EG10407 | 1474 | 54.65 | 36793.07 | 91.2% |
| | alaS-N | Ala-tRNA synthetase | EG10034 | 2685 | 96.87 | 11113.83 | 67.8% |
| | aspS-C | Asp-tRNA synthetase | EG10097 | 1831 | 66.75 | 9117.27 | 53.1% |
| | asnS-N | Asn-tRNA synthetase | EG10094 | 1455 | 53.4 | 18233.11 | 83.4% |
| | leuS-C | Leu-tRNA synthetase | EG10532 | 2641 | 98.07 | 26850.33 | 75.2% |
| | argS-N | Arg-tRNA synthetase | EG10071 | 1788 | 65.48 | 56168.27 | 81.7% |
| | cysS-C | Cys-tRNA synthetase | EG10196 | 1444 | 53.03 | 39718.51 | 86.6% |
| | trpS-C | Trp-tRNA synthetase | EG11030 | 1063 | 38.27 | 7965.79 | 66.5% |
| | pheT-C | Phe-tRNA synthetase B | EG10710 | 2446 | 88.21 | 7615.24 | 88.1% |
| | proS-C | Pro-tRNA synthetase | EG10770 | 1777 | 64.53 | 6653.47 | 78.8% |
| | serS-C | Ser-tRNA synthetase | EG10947 | 1351 | 49.24 | 19594.46 | 93.1% |
| | pheS-N | Phe-tRNA synthetase A | EG10709 | 1038 | 37.66 | 56168.27 | 47.3% |
| | glns-N | Gln-tRNA synthetase | EG10390 | 1719 | 64.31 | 6476.21 | 89.3% |
| | tyrS-C | Tyr-tRNA synthetase | EG11043 | 1333 | 48.36 | 1804 | 84.1% |
| | fmt-C | Met-tRNA formyltransferase | EG11268 | 1006 | 34.97 | 60361.96 | 94.0% |
| | glyS-C | Gly-tRNA synthetase B | EG10410 | 2128 | 77.65 | 75594.36 | 90.9% |
| | hisS-C | His-tRNA synthetase | EG10453 | 1333 | 47.83 | 75594.36 | 65.0% |
| | glyQ-C | Gly-tRNA synthetase A | EG10409 | 970 | 35.6 | 30731.61 | 86.3% |

^{a}TIR (translation initiation rate)

Table S4. Purified proteins for the preparation of Control IET. Protein purification yields and requirements for the assembly of Control IET.

TABLE S4

| | | | Concentration purified | | | | | | For |
|---|---|---|---|---|---|---|---|---|---|
| | | | proteins^{a} | | | | | | 200 |
| | | MW | Effective concentration | | | Needed^{b} | | | μL |
| Gene | Description | (ng/nmol) | ng/μL | nmol/μL | μM | ng/μL | nmol/μL | μM | mixure |
| infA-N | translational initiation | 9070 | 3514.9 | 0.39 | 388 | 898 | 0.0090 | 99.0 | 76.6 |
| | factor 1 | | | | | | | | |
| infB-N | translational initiation | 98190 | 8105.0 | 0.08 | 83 | 403 | 0.0041 | 4.1 | 14.9 |
| | factor 2 | | | | | | | | |
| infC-N | translational initiation | 21370 | 29784.9 | 1.39 | 1394 | 105 | 0.0049 | 4.9 | 1.1 |
| | factor 3 | | | | | | | | |
| tufA-C | translational elongation | 44080 | 31598.5 | 0.72 | 717 | 3526 | 0.0800 | 80.0 | 33.5 |
| | factor Tu | | | | | | | | |
| tsf-C | translational elongation | 31250 | 48035.6 | 1.54 | 1537 | 406 | 0.0130 | 13.0 | 2.5 |
| | factor Ts | | | | | | | | |
| fusA-C | translational elongation | 78420 | 89949.7 | 1.15 | 1147 | 337 | 0.0043 | 4.3 | 1.1 |
| | factor G | | | | | | | | |
| lepA-C | translational elongation | 67400 | 1620.5 | 0.02 | 24 | 12 | 0.0002 | 0.2 | 2.2 |
| | factor 4 | | | | | | | | |
| prfA-N | translational release | 41350 | 4696.3 | 0.11 | 114 | 8 | 0.0002 | 0.2 | 0.5 |
| | factor 1 | | | | | | | | |
| prfB-N | translational release | 42080 | 1827.4 | 0.04 | 43 | 8 | 0.0002 | 0.2 | 1.4 |
| | factor 2 | | | | | | | | |
| prfC-N | translational release | 60410 | 4805.9 | 0.08 | 80 | 42 | 0.0007 | 0.7 | 2.6 |
| | factor 3 | | | | | | | | |
| frr-C | ribosome recycling | 21430 | 49667.8 | 2.32 | 2318 | 343 | 0.0160 | 10.0 | 2.1 |
| | factor | | | | | | | | |

^{a}Protein concentration for each factor in our conditions
^{b}Based on requirements defined at Kazuta, Y., Matsuura, T., Ichihashi, N., et al. (2014) Journal of Bioscience and Bioengineering. 118, 554-557

Table S5. Identified proteins and quantified counts from 34-strain TraMOS. (mean±SEM, n=3).

TABLE S5
34-Strain TraMOS

| Group | Gene name | Mean | SD |
|---|---|---|---|
| IET | EF-Ts | 3453.4 | 22.2 |
| | IF1 | 2414.9 | 321.0 |
| | EF-Tu | 1783.8 | 250.7 |
| | IF2 | 1353.7 | 86.1 |
| | EF-G | 935.1 | 60.4 |
| | IF3 | 711.8 | 52.0 |
| | RRF | 149.7 | 7.5 |
| | RF1 | 95.3 | 15.4 |
| | RF2 | 26.1 | 2.3 |
| | RF3 | 4.2 | |
| | EF4 | 1.0 | 0.1 |
| AAT | tyrS | 877.0 | 64.4 |
| | serS | 523.3 | 38.0 |
| | pheT | 195.7 | 31.0 |
| | aspS | 188.3 | 39.2 |
| | thrS | 169.0 | 36.5 |
| | asnS | 115.4 | 18.2 |
| | glnS | 109.4 | 21.2 |
| | fmt | 80.7 | 23.3 |
| | lysS | 65.4 | 7.0 |
| | gltX | 63.6 | 5.3 |
| | trpS | 61.0 | 12.7 |
| | hisS | 33.8 | 12.2 |
| | proS | 28.5 | 4.3 |
| | metG | 20.7 | 1.4 |
| | glyQ | 12.4 | 5.1 |
| | aspS | 12.3 | 7.5 |
| | alaS | 4.8 | 3.9 |
| | leuS | 4.6 | |
| | pheS | 3.8 | 1.1 |
| | valS | 3.3 | 1.3 |
| | ileS | 3.0 | 0.2 |
| | cysS | 2.0 | 0.9 |
| | argS | N.a | |
| | glyS | N.a | |
| Non TraM | Glucosamine/fructose-6-phosphate aminotransferase, isomerizing OS = Escherichia coli (strain B/BL21-DE3) | 154.0 | 24.3 |
| | GN = ECBD_4303 PE = 3 SV = 1 | | |
| | Peptidylprolyl isomerase FKBP-type OS = Escherichia coli (strain B/BL21-DE3) GN = ECBD_0400 PE = 4 | 141.7 | 63.5 |
| | SV = 1 | | |
| | Bifunctional polymyxin resistance protein ArnA OS = Escherichia coli (strain B/BL21-DE3) | 114.1 | 23.6 |
| | SV = 1GN = ECBD_1404 PE = 3 | | |
| | GTP cyclohydrolase I OS = Escherichia coli (strain B/BL21-DE3) GN = ECBD_1505 PE = 3 SV = 1 | 63.0 | 7.5 |
| | Transcriptional regulator, Crp/Fnr family OS = Escherichia coli (strain B/BL21-DE3) GN = ECBD_0931 | 48.8 | 3.9 |
| | PE = 4 SV = 1 | | |
| | Chaperone protein DnaK OS = Escherichia coli (strain B/BL21-DE3) GN = ECBD_3605 PE = 3 SV = 1 | 38.4 | 2.9 |
| | Formyltetrahydrofolate deformylase OS = Escherichia coli (strain B/BL21-DE3) GN = ECBD_2930 PE = 3 | 34.0 | 1.5 |
| | SV = 1 | | |
| | Pseudouridine synthase OS = Escherichia coli (strain B/BL21-DE3) GN = ECBD_1474 PE = 4 SV = 1 | 31.7 | 1.8 |
| | Ferric uptake regulator, Fur family OS = Escherichia coli (strain B/BL21-DE3) GN = ECBD_2978 PE = 4 | 27.0 | 1.3 |
| | SV = 1 | | |
| | Chaperonin GroEL OS = Escherichia coli (strain B/BL21-DE3) GN = ECBD_3888 PE = 4 SV = 1 | 26.8 | 3.3 |
| | Cell division protein FtsZ OS = Escherichia coli (strain B/BL21-DE3) GN = ECBD_3522 PE = 3 SV = 1 | 23.2 | 10.8 |
| | rRNA (Guanine-N(1)-)-methyltransferase OS = Escherichia coli (strain B/BL21-DE3) GN = ECBD_1819 | 22.6 | 2.4 |
| | PE = 3 SV = 1 | | |
| | Uncharacterized protein OS = Escherichia coli (strain B/BL21-DE3) GN = ECBD_2493 PE = 3 SV = 1 | 22.0 | 4.8 |
| | Glyceraldehyde-3-phosphate dehydrogenase OS = Escherichia coli (strain B/BL21-DE3) GN = ECBD_1865 | 17.8 | 5.1 |
| | PE = 3 SV = 1 | | |
| | Histidinol-phosphatase OS = Escherichia coli (strain B/BL21-DE3) GN = ECBD_1637 PE = 3 SV = 1 | 16.2 | 2.9 |
| | OmpA domain protein transmembrane region-containing protein OS = Escherichia coli (strain B/BL21-DE3) | 15.4 | 5.5 |
| | GN = ECBD_2638 PE = 4 SV = 1 | | |
| | Trigger factor OS = Escherichia coli (strain B/BL21-DE3) GN = ECBD_3221 PE = 3 SV = 1 | 14.6 | 3.2 |
| | 2-oxo-acid dehydrogenase E1 subunit, homodimeric type OS = Escherichia coli (strain B/BL21-DE3) | 14.2 | 9.4 |
| | GN = ECBD_3505 PE = 4 SV = 1 | | |
| | Chorismate mutase OS = Escherichia coli (strain B/BL21-DE3) GN = ECBD_1087 PE = 4 SV = 1 | 13.2 | 3.2 |
| | Alkyl hydroperoxide reductase, F subunit OS = Escherichia coli (strain B/BL21-DE3) GN = ECBD_3046 | 12.1 | 5.1 |
| | PE = 3 SV = 1 | | |
| | Transcriptional regulator, PadR-like family OS = Escherichia coli (strain B/BL21-DE3) GN = ECBD_0671 | 11.6 | 1.9 |
| | PE = 4 SV = 1 | | |
| | Arabinose 5-phosphate isomerase OS = Escherichia coli (strain B/BL21-DE3) PE = 3 SV = 1 | 11.5 | 4.5 |
| | ATP synthase F1, beta subunitOS = Escherichia coli (strain B/BL21-DE3) GN = ECBD_4300 PE = 3 | 10.0 | 7.4 |
| | SV = 1 | | |
| | Lysine decarboxylase OS = Escherichia coli (strain B/BL21-DE3) GN = ECBD_3433 PE = 4 SV = 1 | 9.9 | 1.3 |
| | DNA-directed RNA polymerase, alpha subunit OS = Escherichia coli (strain B/BL21-DE3) GN = ECBD_0456 | 9.8 | 3.8 |
| | PE = 3 SV = 1 | | |
| | Ribonuclease, Rne/Rng family OS = Escherichia coli (strain B/BL21-DE3) GN = ECBD_2516 PE = 3 | 9.7 | 5.4 |
| | SV = 1 | | |
| | NADH-quinone oxidoreductase, E subunit OS = Escherichia coli (strain B/BL21-DE3) GN = ECBD_1376 | 9.5 | 3.0 |
| | PE = 4 SV = 1 | | |
| | UspA domain protein OS = Escherichia coli (strain B/BL21-DE3) GN = ECBD_3045 PE = 4 SV = 1 | 9.3 | 0.2 |
| | Peroxiredoxin OS = Escherichia coli (strain B/BL21-DE3) GN = ECBD_3047 PE = 1 SV = 1 | 9.3 | 1.6 |
| | Virulence factor MviM-like protein OS = Escherichia coli (strain B/BL21-DE3) GN = ECBD_2532 PE = 4 | 8.5 | 3.1 |
| | SV = 1 | | |
| | Transcription termination factor Rho OS = Escherichia coli (strain B/BL21-DE3) GN = ECBD_4257 | 8.4 | 4.8 |
| | PE = 3 SV = 1 | | |
| | Uncharacterized protein OS = Escherichia coli (strain B/BL21-DE3) GN = ECBD_0183 PE = 4 SV = 1 | 8.2 | 3.4 |
| | Enolase OS = Escherichia coli (strain B/BL21-DE3) GN = ECBD_0950 PE = 3 SV = 1 | 7.5 | 4.1 |
| | LPP repeat-containing protein OS = Escherichia coli (strain B/BL21-DE3) GN = ECBD_1968 PE = 4 SV = 1 | 7.3 | 0.9 |
| | Heat shock protein HsIVU, ATPase subunit HsIU OS = Escherichia coli (strain B/BL21-DE3) | 6.5 | 3.5 |
| | GN = ECBD_4093 PE = 3 SV = 1 | | |
| | FeS cluster assembly scaffold IscU OS = Escherichia coli (strain B/BL21-DE3) GN = ECBD_1155 PE = 4 | 6.3 | 1.1 |
| | SV = 1 | | |
| | tRNA (Guanine-N(7)-)-methyltransferase OS = Escherichia coli (strain B/BL21-DE3) GN = ECBD_0799 | 6.3 | 1.8 |
| | PE = 3SV = 1 | | |
| | Histone family protein nucleoid-structuring protein H-NS OS = Escherichia coli (strain B/BL21-DE3) | 6.2 | 2.7 |
| | GN = ECBD_2385 PE = 4 SV = 1 | | |
| | Peptidoglycan-associated lipoprotein OS = Escherichia coli (strain B/BL21-DE3) GN = ECBD_2919 PE = 4 | 5.9 | 1.6 |
| | SV = 1 | | |
| | Pyruvate dehydrogenase complex dihydrolipoamide acetyltransferase OS = Escherichia coli | 5.9 | 3.1 |
| | (strain B/BL21-DE3) GN = ECBD_3504 PE = 4 SV = 1 | | |
| | UDP-N-acetylglucosamine--N-acetylmuramyl-(Pentapeptide) pyrophosphoryl-undecaprenol N- | 5.6 | 2.1 |
| | acetylglucosamine transferase OS | | |
| | Protein RecA OS = Escherichia coli (strain B/BL21-DE3) GN = ECBD_1026 PE = 3 SV = 15.6 | 2.2 | |
| | DNA-directed RNA polymerase, beta subunit OS = Escherichia coli (strain B/BL21-DE3) GN = ECBD_4045 | 5.3 | 7.4 |
| | PE = 3 SV = 1 | | |
| | DNA gyrase subunit OS = Escherichia coli (strain B/BL21-DE3) GN = ECBD_0004 PE = 3 SV = 1 | 5.1 | 4.7 |
| | ATP synthase F1, alpha subunit OS = Escherichia coli (strain B/BL21-DE3) GN = ECBD_4298 PE = 3 SV = 1 | 4.8 | 3.9 |
| | Transcriptional regulator, DeoR family OS = Escherichia coli (strain B/BL21-DE3) GN = ECBD_0319 PE = 4 | 4.6 | 0.8 |
| | SV = 1 | | |
| | Transcriptional regulator, Laci family OS = Escherichia coli (strain B/BL21-DE3) GN = ECBD_2908 PE = 4 | 4.3 | 1.0 |
| | SV = 1 | | |
| | Glutaredoxin-like protein NrdH OS = Escherichia coli (strain B/BL21-DE3) GN = ECBD_1046 PE = 4 SV = 1 | 4.3 | 1.3 |
| | Histidine triad (HIT) protein OS = Escherichia coli (strain B/BL21-DE3) GN = ECBD_2498 PE = 4 SV = 1 | 3.9 | 1.4 |
| | DEAD/DEAH box helicase domain protein OS = Escherichia coli (strain B/BL21-DE3) GN = ECBD_0578 | 3.7 | 0.6 |
| | PE = 3 SV = 1 | | |
| | Regulator of RpoD, Rsd/AlgQ OS = Escherichia coli (strain B/BL21-DE3) GN = ECBD_4037 PE = 3 SV = 1 | 3.7 | 0.6 |
| | Transcriptional regulator NikR, CopG family OS = Escherichia coli (strain B/BL21-DE3) GN = ECBD_0260 | 3.7 | 0.4 |
| | PE = 3 SV = 1 | | |
| | GTPase Era OS = Escherichia coli (strain B/BL21-DE3) GN = era PE = 3 SV = 1 | 3.6 | 0.9 |
| | NAD(+) diphosphatase OS = Escherichia coli (strain B/BL21-DE3) GN = ECBD_4036 PE = 3 SV = 1 | 3.6 | 2.3 |
| | Phosphoglycerate mutase 1 family OS = Escherichia coli (strain B/BL21-DE3) GN = ECBD_2912 PE = 3 | 3.5 | 3.4 |
| | SV = 1 | | |
| | 2-oxoglutarate dehydrogenase, E1 subunit OS = Escherichia coli (strain B/BL21-DE3) GN = ECBD_2935 | 3.4 | 0.8 |
| | PE = 4 SV = 1 | | |
| | Phage-related tail fibre protein-like protein OS = Escherichia coli (strain B/BL21-DE3) GN = ECBD_2754 | 3.3 | 0.7 |
| | PE = 4 SV = 1 | | |
| | Transcriptional regulator, LysR family OS = Escherichia coli (strain B/BL21-DE3) GN = ECBD_1501 PE = 4 | 3.3 | 1.6 |
| | SV = 1 | | |
| | Sporulation domain protein OS = Escherichia coli (strain B/BL21-DE3) GN = ECBD_0359 PE = 4 SV = 1 | 3.3 | 1.2 |
| | Acyl carrier protein OS = Escherichia coli (strain B/BL21-DE3) GN = ECBD_2507 PE = 1 SV = 1 | 3.2 | 2.0 |
| | Formate acetyltransferase OS = Escherichia coli (strain B/BL21-DE3) GN = ECBD_2692 PE = 4 SV = 1 | 3.0 | 0.2 |
| | Cluster of Cold-shock DNA-binding domain protein OS = Escherichia coli (strain B/BL21-DE3) | 3.0 | 1.0 |
| | GN = ECBD_2605 PE = 4 SV = 1 | | |
| | Histone family protein DNA-binding protein OS = Escherichia coli (strain B/BL21-DE3) GN = ECBD_3215 | 3.0 | 1.0 |
| | PE = 4 SV = 1 | | |
| | Molybdenum cofactor biosynthesis protein C OS = Escherichia coli (strain B/BL21-DE3) GN = ECBD_2840 | 3.0 | 1.7 |
| | PE = 3 SV = 1 | | |
| | Thioredoxin OS = Escherichia coli (strain B/BL21-DE3) GN = ECBD_4259 PE = 4 SV = 1 | 3.0 | 0.9 |
| | Succinate dehydrogenase flavoprotein subunit OS = Escherichia coli (strain B/BL21-DE3) GN = ECBD_2937 | 3.0 | 1.6 |
| | PE = 3 SV = 1 | | |
| | Carbohydrate kinase, thermoresistant glucokinase family OS = Escherichia coli (strain B/BL21-DE3) | 2.9 | 2.4 |
| | GN = ECBD_0306 PE = 4 SV = 1 | | |
| | Chaperone protein DnaJ OS = Escherichia coli (strain B/BL21-DE3) GN = ECBD_3604 PE = 3 SV = 1 | 2.8 | 2.3 |
| | Pyruvate formate-lyase-activating enzyme OS = Escherichia coli (strain B/BL21-DE3) GN = ECBD_2693 | 2.7 | 1.6 |
| | PE = 3 SV = 1 | | |
| | ProQ activator of osmoprotectant transporter ProP OS = Escherichia coli (strain B/BL21-DE3) | 2.7 | 1.3 |
| | GN = ECBD_1809 PE = 3 SV = 1 | | |
| | Apolipoprotein N-acyltransferase OS = Escherichia coli (strain B/BL21-DE3) GN = ECBD_2994 PE = 3 | 2.7 | 0.6 |
| | SV = 1 | | |
| | Peptidylprolyl isomerase FKBP-type OS = Escherichia coli (strain B/BL21-DE3) GN = ECBD_3822 PE = 4 | 2.6 | 1.4 |
| | SV = 1 | | |
| | Transcriptional regulator, DeoR family OS = Escherichia coli (strain B/BL21-DE3) GN = ECBD_4143 PE = 4 | 2.6 | 1.4 |
| | SV = 1 | | |
| | YodA domain protein OS = Escherichia coli (strain B/BL21-DE3) GN = ECBD_1672 PE = 4 SV = 1 | 2.4 | 0.7 |
| | Acetyltransferase OS = Escherichia coli (strain B/BL21-DE3) GN = ECBD_1620 PE = 4 SV = 1 | 2.3 | 0.4 |
| | KpsF/GutQ family protein OS = Escherichia coli (strain B/BL21-DE3) GN = ECBD_0545 PE = 4 SV = 1 | 2.3 | 1.1 |
| | Formate acetyltransferase OS = Escherichia coli (strain B/BL21-DE3) GN = ECBD_0626 PE = 4 SV = 1 | 2.3 | 1.4 |
| | Phosphoenolpyruvate-protein phosphotransferase OS = Escherichia coli (strain B/BL21-DE3) | 2.3 | 1.4 |
| | GN = ECBD_1265 PE = 4 SV = 1 | | |
| | Uncharacterized protein OS = Escherichia coli (strain B/BL21-DE3) GN = ECBD_4273 PE = 4 SV = 1 | 2.0 | 0.1 |
| | Uncharacterized protein OS = Escherichia coli (strain B/BL21-DE3) GN = ECBD_3976 PE = 4 SV = 1 | 2.0 | 0.9 |
| | Putative transferase OS = Escherichia coli (strain B/BL21-DE3) GN = ECBD_0472 PE = 4 SV = 1 | 1.9 | 1.5 |
| | Tyrosine recombinase XerD OS = Escherichia coli (strain B/BL21-DE3) GN = ECBD_0843 PE = 3 SV = 1 | 1.9 | 1.5 |
| | Cytochrome bd ubiquinol oxidase subunit I OS = Escherichia coli (strain B/BL21-DE3) GN = ECBD_2928 | 1.9 | 1.5 |
| | PE = 4 SV = 1 | | |
| | Dihydrolipoamide dehydrogenase OS = Escherichia coli (strain B/BL21-DE3) GN = ECBD_3503 PE = 4 | 1.9 | 1.5 |
| | SV = 1 | | |
| | L-serine dehydratase 1 OS = Escherichia coli (strain B/BL21-DE3) GN = ECBD_0931 PE = 4 SV = 1 | 1.9 | 2.4 |
| | NAD-dependent epimerase/dehydratase OS = Escherichia coli (strain B/BL21-DE3) GN = ECBD_2726 | 1.7 | 0.7 |
| | PE = 4 SV = 1 | | |
| | Sulfatase OS = Escherichia coli (strain B/BL21-DE3) GN = ECBD_4069 PE = 4 SV = 1 | 1.7 | 0.6 |
| | Primosomal replication priB and priB OS = Escherichia coli (strain B/BL21-DE3) GN = ECBD_3189 PE = 4 | 1.7 | 0.5 |
| | SV = 1 | | |
| | Iron-sulfur cluster assembly accessory protein OS = Escherichia coli (strain B/BL21-DE3) GN = ECBD_3462 | 1.7 | 0.5 |
| | PE = 3 SV = 1 | | |
| | Chaperone protein HtpG OS = Escherichia coli (strain B/BL21-DE3) GN = ECBD_3183 PE = 3 SV = 1 | 1.6 | 1.0 |
| | 3-oxoacyl-(Acyl-carrier-protein) reductase OS = Escherichia coli (strain B/BL21-DE3) GN = ECBD_2508 | 1.4 | 0.7 |
| | PE = 4 SV = 1 | | |
| | 4-hydroxy-3-methylbut-2-en-1-yl diphosphate synthase (flavodoxin) OS = Escherichia coli | 1.4 | 0.7 |
| | (strain B/BL21-DE3) GN = ECBD_1171 PE = 3 SV = 1 | | |
| | DNA-directed RNA polymerase, beta subunit OS = Escherichia coli (strain B/BL21-DE3) GN = ECBD_4046 | 1.4 | 0.7 |
| | PE = 3 SV = 1 | | |
| | Cold-shock DNA-binding domain protein OS = Escherichia coli (strain B/BL21-DE3) GN = ECBD_1818 | 1.3 | 0.6 |
| | PE = 4 SV = 1 | | |
| | Inorganic diphosphatase OS = Escherichia coli (strain B/BL21-DE3) GN = ECBD_3807 PE = 3 | 1.3 | 0.5 |
| | SV = 1 | | |
| | Peptidyl-prolyl cis-trans isomerase cyclophilin type OS = Escherichia coli (strain B/BL21-DE3) | 1.3 | 0.5 |
| | GN = ECBD_3133 PE = 4 SV = 1 | | |
| | Iron-containing alcohol dehydrogenase OS = Escherichia coli (strain B/BL21-DE3) GN = ECBD_4078 | 1.3 | 0.5 |
| | PE = 4 SV = 1 | | |
| | Acetylornithine deacetylase (ArgE) OS = Escherichia coli (strain B/BL21-DE3) GN = ECBD_4067 | 1.3 | 0.5 |
| | PE = 3 SV = 1 | | |
| | IscR-regulated protein YhgI OS = Escherichia coli (strain B/BL21-DE3) GN = ECBD_0331 PE = 3 SV = 1 | 1.0 | 0.1 |
| | Superoxide dismutase OS = Escherichia coli (strain B/BL21-DE3) GN = ECBD_1987 PE = 4 SV = 1 | 1.0 | 0.1 |
| | Transcriptional regulator, AsnC family OS = Escherichia coli (strain B/BL21-DE3) GN = ECBD_2706 | 1 .0 | 0.1 |
| | PE = 4 SV = 1 | | |
| | (Acyl-carrier-protein) phosphodiesterase OS = Escherichia coli (strain B/BL21-DE3) GN = ECBD_3257 | 1.0 | 0.1 |
| | PE = 3 SV = 1 | | |
| | 6-phosphogluconate dehydrogenase, decarboxylating OS = Escherichia coli (strain B/BL21-DE3) | 1.0 | 0.1 |
| | GN = ECBD_1630 PE = 4 SV = 1 | | |
| | ATP-dependent chaperone CIpB OS = Escherichia coli (strain B/BL21-DE3) GN = ECBD_1092 PE = 4 | 1.0 | 0.1 |
| | SV = 1 | | |
| | ATP-NAD/AcoX kinase OS = Escherichia coli (strain B/BL21-DE3) GN = ECBD_1072 PE = 3 SV = 1 | 1.0 | 0.1 |
| | Catalase/peroxidase HPI OS = Escherichia coli (strain B/BL21-DE3) GN = ECBD_4081 PE = 3 SV = 1 | 1.0 | 0.1 |
| | L-asparaginase, type I OS = Escherichia coli (strain B/BL21-DE3) GN = ECBD_1877 PE = 4 SV = 1 | 1.0 | 0.1 |
| Ribosomal | Ribosomal protein L17 OS = Escherichia coli (strain B/BL21-DE3) GN = ECBD_0457 PE = 3 SV = 1 | 42.0 | 8.6 |
| Proteins | Ribosomal protein L33 OS = Escherichia coli (strain B/BL21-DE3) GN = ECBD_0090 PE = 3 SV = 1 | 17.7 | 7.2 |
| | Ribosomal protein S10 OS = Escherichia coli (strain B/BL21-DE3) GN = ECBD_0430 PE = 3 SV = 1 | 13.1 | 3.4 |
| | Ribosomal protein L6 OS = Escherichia coli (strain B/BL21-DE3) GN = ECBD_0446 PE = 3 SV = 1 | 12.0 | 7.1 |
| | Ribosomal protein L7/L12 OS = Escherichia coli (strain B/BL21-DE3) GN = ECBD_4047 PE = 3 SV = 1 | 10.7 | 6.7 |
| | Ribosomal protein S20 OS = Escherichia coli (strain B/BL21-DE3) GN = ECBD_3593 PE = 3 SV = 11 | 10.4 | 5.4 |
| | Ribosomal protein L27 OS = Escherichia coli (strain B/BL21-DE3) GN = ECBD_0557 PE = 3 SV = 1 | 8.0 | 1.6 |
| | Ribosomal proteirt S21 OS = Escherichia coli (strain B/BL21-DE3) GN = ECBD_0676 PE = 3 SV = 1 | 7.5 | 5.1 |
| | Ribosomal protein L11 OS = Escherichia coli (strain B/BL21-DE3) GN = ECBD_0450 PE = 3 SV = 1 | 6.6 | 2.4 |
| | Ribosomal protein S2 OS = Escherichia coli (strain B/BL21-DE3) GN = ECBD_03450 PE = 3 SV = 1 | 5.5 | 3.3 |
| | Ribosomal protein L35 OS = Escherichia coli (strain B/BL21-DE3) GN = ECBD_1928 PE = 3 SV = 1 | 5.2 | 3.2 |
| | 50S Ribosomal protein L10 OS = Escherichia coli (strain B/BL21-DE3) GN = ECBD_4048 PE = 3 SV = 1 | 4.6 | 1.3 |
| | Ribosomal protein L14 OS = Escherichia coli (strain B/BL21-DE3) GN = ECBD_0441 PE = 3 SV = 1 | 4.3 | 2.2 |
| | Ribosomal protein L24 OS = Escherichia coli (strain B/BL21-DE3) GN = ECBD_0442 PE = 3 SV = 1 | 4.0 | 0.8 |
| | Ribosomal proteirt L4/L1e OS = Escherichia coli (strain B/BL21-DE3) GN = ECBD_0432 PE = 3 SV = 1 | 3.9 | 1.4 |
| | Ribosomal protein S1 OS = Escherichia coli (strain B/BL21-DE3) GN = ECBD_2684 PE = 4 SV = 1 | 3.9 | 2.3 |
| | Ribosomal protein S4 OS = Escherichia coli (strain B/BL21-DE3) GN = ECBD_0455 PE = 3 SV = 1 | 3.9 | 2.3 |
| | Ribosomal protein S3 OS = Escherichia coli (strain B/BL21-DE3) GN = ECBD_0437 PE = 3 SV = 1 | 3.9 | 3.3 |
| | Ribosomal protein S6 OS = Escherichia coli (strain B/BL21-DE3) GN = ECBD_3834 PE = 3 SV = 1 | 3.7 | 2.3 |
| | Ribosomal protein L25 OS = Escherichia coli (strain B/BL21-DE3) GN = ECBD_1472 PE = 3 SV = 1 | 3.6 | 1.8 |
| | Ribosomal protein L9 OS = Escherichia coli (strain B/BL21-DE3) GN = ECBD_3831 PE = 3 SV = 1 | 3.2 | 2.3 |
| | Ribosomal protein S15 OS = Escherichia coli (strain B/BL21-DE3) GN = ECBD_0575 PE = 3 SV = 1 | 3.2 | 3.7 |
| | Ribosomal proteirt L5 OS = Escherichia coli (strain B/BL21-DE3) GN = ECBD_0443 PE = 3 SV = 1 | 2.9 | 1.8 |
| | Ribosomal protein L16 OS = Escherichia coli (strain B/BL21-DE3) GN = ECBD_0438 PE = 3 SV = 1 | 2.9 | 1.5 |
| | Ribosomal protein L32 OS = Escherichia coli (strain B/BL21-DE3) GN = ECBD_2512 PE = 3 SV = 1 | 2.9 | 1.8 |
| | Ribosomal protein L20 OS = Escherichia coli (strain B/BL21-DE3) GN = ECBD_1929 PE = 3 SV = 1 | 2.9 | 2.4 |
| | Ribosomal protein S5 OS = Escherichia coli (strain B/BL21-DE3) GN = ECBD_0448 PE = 3 SV = 1 | 2.9 | 2.4 |
| | Ribosomal protein L29 OS = Escherichia coli (strain B/BL21-DE3) GN = ECBD_0439 PE = 3 SV = 1 | 2.6 | 1.4 |
| | Ribosomal protein S16 OS = Escherichia coli (strain B/BL21-DE3) GN = ECBD_1077 PE = 3 SV = 1 | 2.6 | 0.9 |
| | Ribosomal protein L1 OS = Escherichia coli (strain B/BL21-DE3) GN = ECBD_4049 PE = 3 SV = 1 | 2.6 | 1.4 |
| | Ribosomal protein L26 OS = Escherichia coli (strain B/BL21-DE3) GN = ECBD_0089 PE = 3 SV = 1 | 2.3 | 0.4 |
| | Ribosomal protein L18 OS = Escherichia coli (strain B/BL21-DE3) GN = ECBD_0447 PE = 3 SV = 1 | 2.3 | 1.3 |
| | Ribosomal protein L13 OS = Escherichia coli (strain B/BL21-DE3) GN = ECBD_0516 PE = 3 SV = 1 | 2.2 | 2.1 |
| | Ribosomal protein S12 OS = Escherichia coli (strain B/BL21-DE3) GN = ECBD_0407 PE = 3 SV = 1 | 2.0 | 0.9 |
| | Ribosomal protein L19 OS = Escherichia coli (strain B/BL21-DE3) GN = ECBD_1080 PE = 3 SV = 1 | 1.6 | 1.0 |
| | Ribosomal protein L15 OS = Escherichia coli (strain B/BL21-DE3) GN = ECBD_0450 PE = 3 SV = 1 | 1.0 | 0.1 |
| | Ribosomal protein S13 OS = Escherichia coli (strain B/BL21-DE3) GN = ECBD_0453 PE = 3 SV = 1 | 1.0 | 0.1 |
| | Ribosomal protein S17 OS = Escherichia coli (strain B/BL21-DE3) GN = ECBD_0440 PE = 3 SV = 1 | 1.0 | 0.1 |

Table S7. Detailed strain composition of 34-strain TraMOS A and B consortia. See Supplementary Information Section 3.1.4 and FIG. 2E.

TABLE S7

| | 34-strain | 34-strain |
|---|---|---|
| | A | B |
| Strain # | 34 | 34 |
| Name | Relative density (%) | |
| 1Tg IF1 | 11.98 | 13.79 |
| 1Tg IF2 | 10.28 | 11.83 |
| 1Tg IF3 | 4.43 | 5.10 |
| 1Tg EF-G | 2.25 | 2.59 |
| 1Tg EF-Tu | 15.84 | 18.23 |
| 1Tg EF-Ts | 23.28 | 26.79 |
| 1Tg EF4 | 1.33 | 1.53 |
| 1Tg RF1 | 0.35 | 0.41 |
| 1Tg RF2 | 0.30 | 0.35 |
| 1Tg RF3 | 3.54 | 4.08 |
| 1Tg RRF | 0.21 | 0.24 |
| 1Tg alaS | 9.12 | 5.25 |
| 1Tg argS | 0.49 | 0.28 |
| 1Tg asnS | 1.74 | 1.00 |
| 1Tg aspS | 2.36 | 1.36 |
| 1Tg cysS | 0.47 | 0.27 |
| 1Tg glnS | 0.83 | 0.48 |
| 1Tg gltX | 0.47 | 0.27 |
| 1Tg glyQ | 0.14 | 0.08 |
| 1Tg glyS | 0.13 | 0.07 |
| 1Tg hisS | 0.42 | 0.24 |
| 1Tg ileS | 1.17 | 0.67 |
| 1Tg leuS | 0.20 | 0.12 |
| 1Tg lysS | 0.38 | 0.22 |
| 1Tg fmt | 0.49 | 0.28 |
| 1Tg metG | 2.33 | 1.34 |
| 1Tg pheS | 0.13 | 0.07 |
| 1Tg pheT | 2.18 | 1.26 |
| 1Tg proS | 2.27 | 1.30 |
| 1Tg serS | 0.14 | 0.08 |
| 1Tg thrS | 0.16 | 0.09 |
| 1Tg trpS | 0.18 | 0.11 |
| 1Tg tyrS | 0.28 | 0.16 |
| 1Tg valS | 0.11 | 0.06 |

Table S9. Detailed strain composition of 15-strain TraMOS consortia. See Supplementary Information Section 3.3 and FIG. 3.

TABLE S9

| 3Tg strains coding three TraM gene | | | | | |
|---|---|---|---|---|---|
| TraM genes coded in | | pIURAH | | | |
| | | pIURCM | | | |
| | | pIURKL | | | |
| | | | | Relative density (%) | |
| Strain name | Coding for | | | A | B |
| 3TgEF | EF-Tu | EF-G | EF-Ts | 55.83 | 56.75 |
| 2Tg IET 3 | IF1 | — | IF2 | 18.61 | 18.92 |
| 3TgRF | RF1 | RF3 | RF2 | 0.62 | 0.63 |
| 3TgE4RRF | EF4 | RRF | EF-G | 9.93 | 10.09 |
| 2Tg IET 6 | EF-G | — | IF3 | 4.34 | 4.41 |
| 1Tg EF-G | EF-G | — | — | 7.44 | 7.57 |
| 2TgAAT 4 | leuS | hisS | fmt | 0.517 | 0.263 |
| 3TgAAT1 | metG | gltX | aspS | 0.388 | 0.197 |
| 3TgAAT2 | ileS | glnS | trpS | 0.310 | 0.158 |
| 3TgAAT3 | pheT | pheS | lysS | 0.284 | 0.145 |
| 3TgAAT4 | proS | asnS | valS | 0.233 | 0.118 |
| 3TgAAT5 | alaS | alaS | serS | 1.379 | 0.701 |
| 3TgAAT6 | tyrS | argS | glyQ | 0.052 | 0.026 |
| 3TgAAT7 | glyS | thrS | cysS | 0.031 | 0.016 |
| 3TgAAT8 | glyS | — | thrS | 0.031 | 0.016 |

Table S10. Cys-proteases inhibitors used in multiple sequence alignment. 3PNR_B corresponds to the PbICP inhibitor crystallized with a Cys-protease Falcipain-2⁷. See Fig. S10B for details.

TABLE S10

| Accession | | |
|---|---|---|
| number | Name | Species |
| CAC39242.1 | chagasin | Trypanosoma cruzi |
| XP_813685.1 | cysteine peptidase inhibitor | Trypanosoma cruzi strain CL Brener |
| XP_001683694.1 | inhibitor of cysteine peptidase | Leishmania major strain Friedlin |
| XP_011775991.1 | ICP, putative | Trypanosoma brucei gambiense DAL972 |
| XP_847475.1 | inhibitor of cysteine peptidase | Trypanosoma brucei brucei TREU927 |
| XP_003392585.1 | inhibitor of cysteine peptidase | Leishmania infantum JPCM5 |
| XP_010699513.1 | inhibitor of cysteine peptidase | Leishmania panamensis |
| XP_001565448.1 | inhibitor of cysteine peptidase | Leishmania braziliensis MHOM/BR/75/M2904 |
| XP_008859391.1 | cysteine protease inhibitor 1 (EhlCP1), putative | Entamoeba nuttalli P19 |
| XP_653255.1 | cysteine protease inhibitor 1 (EhlCP1) | Entamoeba histolytica HM-1:IMSS |
| XP_003875994.1 | inhibitor of cysteine peptidase | Leishmania mexicana MHOM/GT/2001/U1103 |
| WP_034012977.1 | peptidase inhibitor | Pseudomonas aeruginosa |
| WP_003241568.1 | peptidase inhibitor | Pseudomonas mendocina |
| WP_000604089.1 | peptidase inhibitor | Bacillus cereus |
| 3PNR_B | PblCP-C (crystal structure with Falcipain-2 | Plasmodium berghei |
| | protease from Plasmodium falciparium) | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A microbial culture comprising a plurality of microbial strains, each strain comprising a different recombinant plasmid including a gene encoding a different protein involved in translation of mRNA, wherein the protein expression level of each protein is controlled to a pre-defined level, such that the proteins are capable of forming a multi-protein complex which translates an mRNA molecule into a polypeptide in a reaction mixture.

2. The microbial culture of claim 1, wherein the amount of each protein in the microbial culture is determined by:
(a) the density of the microbial strain in the culture,
(b) the copy number of the plasmid comprising the gene encoding the protein,
(c) the sequence of the ribosomal binding site in the gene encoding the protein; or
(d) a combination of (a), (b) and (c).

3. The microbial culture of claim 1, wherein each gene has the same promoter.

4. The microbial culture of claim 3, wherein the promoter is a PT7/lacO hybrid promoter.

5. The microbial culture of claim 1, wherein the microbial culture comprises E. coli.

6. The microbial culture of claim 1, wherein each protein includes a tag to facilitate isolation of the protein.

7. (canceled)

8. The microbial culture of claim 1, wherein each microbial strain comprises a single plasmid including a gene encoding a protein involved in translation of mRNA.

9. The microbial culture of claim 1, wherein at least one strain comprises more than one plasmid including a gene encoding a protein involved in translation of mRNA.

10. The microbial culture claim 1, wherein the proteins comprise initiation factors, elongation factors, termination/release factors, a ribosome recycling factor and tRNA-Amino acyl-transferases.

11. The microbial culture of claim 10, wherein:
(a) the initiation factors are translational initiation factor 1, translational initiation factor 2, and translational initiation factor 3;
(b) the elongation factors are translational elongation factor G, translational elongation factor Tu, translational elongation factor Ts, and translational elongation factor 4;
(c) the termination/release factors are translational release factor 1, translational release factor 2, and translational release factor 3; and
(d) the tRNA-Amino acyl-transferases are Val-tRNA synthetase, Met-tRNA synthetase, Ile-tRNA synthetase, Thr-tRNA synthetase, Lys-tRNA synthetase, Glu-tRNA synthetase, Ala-tRNA synthetase, Asp-tRNA synthetase, Asn-tRNA synthetase, Leu-tRNA synthetase, Arg-tRNA synthetase, Cys-tRNA synthetase, Trp-tRNA synthetase, Phe-tRNA synthetase B, Pro-tRNA synthetase, Ser-tRNA synthetase, Phe-tRNA synthetase A, Gln-tRNA synthetase, Tyr-tRNA synthetase, Met-tRNA formyltransferase, Gly-tRNA synthetase B, His-tRNA synthetase, and Gly-tRNA synthetase A.

12. A method of making a multi-protein complex which is capable of translating an mRNA molecule into a polypeptide in a reaction mixture, the method comprising:
(a) providing a microbial culture comprising a plurality of microbial strains, each strain comprising a different recombinant plasmid including a gene encoding a different protein involved in translation of mRNA, wherein the protein expression level of each protein is controlled to a pre-defined level, such that the proteins are capable of forming a multi-protein complex; and
(b) simultaneously isolating the proteins from the microbial culture, thereby forming the multi-protein complex.

13. The method of claim 12, wherein the microbial culture comprises E. coli.

14. The method of claim 12, wherein each protein includes a tag to facilitate isolation of the protein.

15. The method of claim 14, wherein the tag is a poly His tag.

16. The method of claim 12, wherein the proteins comprise initiation factors, elongation factors, termination/release factors, a ribosome recycling factor and tRNA-Amino acyl-transferases.

17. The method of claim 16, wherein:
(a) the initiation factors are translational initiation factor 1, translational initiation factor 2, and translational initiation factor 3;
(b) the elongation factors are translational elongation factor G, translational elongation factor Tu, translational elongation factor Ts, and translational elongation factor 4;
(c) the termination/release factors are translational release factor 1, translational release factor 2, and translational release factor 3; and
(d) the tRNA-Amino acyl-transferases are Val-tRNA synthetase, Met-tRNA synthetase, Ile-tRNA synthetase, Thr-tRNA synthetase, Lys-tRNA synthetase, Glu-tRNA synthetase, Ala-tRNA synthetase, Asp-tRNA synthetase, Asn-tRNA synthetase, Leu-tRNA synthetase, Arg-tRNA synthetase, Cys-tRNA synthetase, Trp-tRNA synthetase, Phe-tRNA synthetase B, Pro-tRNA synthetase, Ser-tRNA synthetase, Phe-tRNA synthetase A, Gln-tRNA synthetase, Tyr-tRNA synthetase, Met-tRNA formyltransferase, Gly-tRNA synthetase B, His-tRNA synthetase, and Gly-tRNA synthetase A.

18. A method of translating an mRNA molecule into a polypeptide, the method comprising:
(a) providing a microbial culture comprising a plurality of microbial strains, each strain comprising a different recombinant plasmid comprising a gene encoding a different protein involved in translation of mRNA, wherein the protein expression level of each protein is controlled to a pre-defined level, such that the proteins are capable of forming a multi-protein complex which translates an mRNA molecule into a polypeptide in a reaction mixture;
(b) simultaneously isolating the proteins from the microbial culture, thereby forming the multi-protein complex;
(c) forming a reaction mixture comprising the multi-protein complex, amino acids, ribosomes, and the mRNA molecule or a DNA molecule encoding the mRNA;
(d) incubating the reaction mixture under conditions suitable for translation of the mRNA molecule into a polypeptide; and
(e) isolating the polypeptide.

19. The method of claim 18, wherein the multi-protein complex comprises initiation factors, elongation factors, termination/release factors, a ribosome recycling factor and tRNA-Amino acyl-transferases.

20. The method of claim 19, wherein:
(a) the initiation factors are translational initiation factor 1, translational initiation factor 2, and translational initiation factor 3;
(b) the elongation factors are translational elongation factor G, translational elongation factor Tu, translational elongation factor Ts, and translational elongation factor 4;
(c) the termination/release factors are translational release factor 1, translational release factor 2, and translational release factor 3; and
(d) the tRNA-Amino acyl-transferases are Val-tRNA synthetase, Met-tRNA synthetase, Ile-tRNA synthetase, Thr-tRNA synthetase, Lys-tRNA synthetase, Glu-tRNA synthetase, Ala-tRNA synthetase, Asp-tRNA synthetase, Asn-tRNA synthetase, Leu-tRNA synthetase, Arg-tRNA synthetase, Cys-tRNA synthetase, Trp-tRNA synthetase, Phe-tRNA synthetase B, Pro-tRNA synthetase, Ser-tRNA synthetase, Phe-tRNA synthetase A, Gln-tRNA synthetase, Tyr-tRNA synthetase, Met-tRNA formyltransferase, Gly-tRNA synthetase B, His-tRNA synthetase, and Gly-tRNA synthetase A.

21. The method of claim 18, wherein each protein includes a tag to facilitate isolation of the protein and the step of isolating the polypeptide is carried out by contacting the reaction mixture comprising the polypeptide with a solid support that specifically binds the tag, thereby separating the polypeptide from the proteins involved in translation of mRNA.

22. (canceled)
